(19) United States
(12) Patent Application Publication
(10) Pub. No.: US 2022/0226447 A1
(43) Pub. Date: Jul. 21, 2022

(54) ANTI-ABETA VACCINE THERAPY

(71) Applicant: AC Immune SA, Lausanne (CH)

(72) Inventors: Andrea Pfeifer, Lausanne (CH); Andreas Muhs (Deceased), (US)

(21) Appl. No.: 17/612,921

(22) PCT Filed: May 20, 2020
(86) PCT No.: PCT/EP2020/064172

§ 371 Date: Nov. 19, 2021

(30) Foreign Application Priority Data
May 21, 2019 (EP) 19175810.1
Jul. 10, 2019 (EP) 19185593.1
Apr. 27, 2020 (EP) 20171549.7
Apr. 29, 2020 (EP) 20172205.5

(51) Int. Cl. A61K 39/00; A61K 39/39; A61P 25/28

(52) U.S. Cl. CPC A61K 39/0007; A61K 39/39; A61K 2039/545; A61K 2039/6018; A61K 2039/55572; A61P 25/28

(57) ABSTRACT

A liposomal vaccine composition comprising: a. A β-amyloid (Aβ)-derived peptide antigen displayed on the surface of the liposome that comprises, consists essentially of or consists of amino acids 1-15 of Aβ, b. An adjuvant comprising monophosphoryl lipid A (MPLA) is used for inducing an anti-Aβ immune response in a human subject without inducing a serious adverse event. The β-amyloid (Aβ)-derived peptide antigen (SEQ ID NO: 1) is administered in an amount of 300-2000 μg, preferably around 1000 μg. The MPLA is administered in an amount of 15-600 μg, preferably around 175 μg. The liposomal vaccine composition is administered intramuscularly or subcutaneously.

### FIELD OF THE INVENTION

The invention relates to anti-abeta therapeutic vaccines and their use in inducing an anti-Aβ immune response without inducing serious adverse events. Such vaccines are useful for the treatment and prevention of diseases, in particular an amyloid-beta associated disease or condition or a condition characterised by, or associated with, loss of cognitive memory capacity, such as Alzheimer's disease (AD) and Down syndrome (DS), including Down syndrome-related Alzheimer's disease. The vaccines incorporate Aβ-derived peptide antigens on an outer surface of a liposome.

### BACKGROUND

Alzheimer's Disease (AD) is a devastating, progressive degenerative disorder characterized by loss of cognitive functions, including memory, as well as the loss of ability to perform regular daily activities. AD affects approximately 40 million patients worldwide, with the number increasing rapidly as the population ages. The major neuropathological change in the brain of AD patients is neuronal death, mainly in memory and cognition-related regions (Soto, 1999). One of the most striking pathological features of AD is the abundant presence of amyloid beta (abeta, Abeta, β-amyloid, Aβ) plaques in brains of diseased individuals (Soto, 1999). Aβ plaques are formed by the 39 to 43 amino acid long Aβ peptide, which is in random coil conformation in its natural non-pathological form. During the transition to the pathological state, it transforms mainly into a β-sheet secondary structure, spontaneously aggregating into insoluble deposits.

The few currently available treatments for AD are considered to be primarily symptomatic in their action. Despite significant efforts put into developing treatments over the years, no disease modifying treatment for AD has been approved to date. Attempts have been made in order to develop an immunotherapeutic that would neutralize pathological Aβ in the diseased brain over the long term (Winblad, 2014). Vaccines present the advantage of stimulating the immune system to produce a pool of slightly different, but very specific antibodies, while the response can be further recalled by additional vaccinations, if needed.

However, an active immunization (vaccination) approach against Aβ represents several main challenges. Amyloid beta is a so-called self-antigen, which the human body is constantly exposed to. Therefore, it is quite difficult to break immune tolerance and induce an antibody response against it. In addition, it is quite difficult to induce a strong immune response to a vaccine in elderly and sick people, such as AD patients, due to their weakened immune system and decreased number of immune cells.

In a reported initial study, a full-length Aβ1-42 vaccine (AN1792) induced an antibody response and a promising efficacy, with a slower rate of cognitive decline in patients who had received vaccination than in placebo-treated patients (Gilman, 2005). However, 6% of treated patients developed meningoencephalitis, an inflammatory reaction considered to be due to a T-cell-mediated response against full length Aβ1-42 (Orgogozo, 2003).

Another known anti-Aβ vaccine, ACI-24, contains a sequence of 15-amino acids with complete identity with the human sequence 1-15 of Aβ (WO2007/068411). This peptide antigen is linked to a liposomal carrier with the aim to stimulate antibodies against Aβ, while avoiding meningoencephalitis and hemorrhage (Muhs, 2007, Pihlgren, 2013). The choice of the Aβ1-15 peptide serving as the antigen was based on the rationale that this sequence contains a B-cell epitope, but lacks a strong T-cell reactive site of full-length Aβ1-42 (Monsonego, 2003), the latter being considered to be the cause of the unwanted inflammatory reactions. ACI-24 has been shown to act through a simultaneous activation of a B-cell receptor specific for Aβ1-15 and the Toll-like receptor 4 (TLR4), the latter activated by monophosphoryl lipid A (MPLA) adjuvant present in the ACI-24 vaccine (Pihlgren, 2013). B-cells are activated to proliferate and produce immunoglobulin (Ig) by cross-linking the B-cell surface Ig receptor.

Down syndrome (DS), also known as trisomy 21, is one of the most common causes of intellectual disability, affecting 1 in 800 newborns. This condition most commonly involves triplication of chromosome 21 (Belichenko, 2016). Subjects with DS have characteristic facial features, deficits in the immune and endocrine systems, and delayed cognitive development. Major improvements in medical care and understanding of the condition have not only improved the quality of life for DS subjects, but have also significantly extended their lifespan. DS subjects now have comparable mortality rates up to age 35 to those with other intellectual disabilities. However, after age 35, the mortality rate doubles every 6.4 years for DS subjects versus 9.6 years for non-DS people. An average life expectancy for DS subjects is 60 years, compared to an average of 79 years for the general population in the USA.

A key feature of adult subjects with DS is their increased risk of developing similar clinical symptoms of Alzheimer's Disease (AD), characterized by a decline in specific cognitive domains suggestive of a diagnosis of dementia. Virtually all subjects with DS older than 40 years exhibit neuropathological changes similar to AD, in the form of senile plaque formation and neurofibrillary tangles (Head, 2012). It is well accepted that the neuropathology for AD-like cognitive decline involves the β-amyloid (Aβ) peptide deposition and subsequent plaque formation, neurofibrillary tangles, vascular damage, neuro-inflammation and ultimately neuronal cell death. The gene of the amyloid protein precursor (APP), which encodes the precursor protein of Aβ, resides on chromosome 21. In subjects with DS, the entire or at least a part of chromosome 21 is present in triplicate. Consequently, this leads to three copies of the gene that encodes APP, which results in the generation of an excess of Aβ. An increased Aβ protein production, has been shown to correlate with AD-like symptoms in DS subjects as well as in the general population that develops AD (Head, 2012). These findings show conclusively that lifelong overexpression of wild-type APP causes cognitive decline in subjects with DS, in a similar way to the amyloid cascade hypothesis used to describe subjects with AD. Down syndrome-related Alzheimer's Disease is characterized by the presence of brain neuropathological hallmarks of Alzheimer's Disease (including notably the accumulation of brain amyloid plaques and neurofibrillary tangles) which can lead, when the brain lesions are sufficiently developed, to the appearance of clinical symptoms like cognitive decline and functional impairment.

The decline in cognitive function for DS subjects occurs over the years prior to a dementia diagnosis. Cognitive decline is classified into three categories: mild, moderate, and severe. Mild cognitive decline is often characterized by noticeable memory lapses that impact daily life as well as behavioral changes. Moderate cognitive decline is characterized by increased memory loss that extends farther into the past, significant personality changes caused by agitation and confusion, changes in sleep patterns, and a need for assistance in daily life. Severe cognitive decline can mean losing the ability to communicate, a severe decline in physical capabilities, and a need for full-time help with routine daily tasks. Symptoms such as apraxia and agnosia are reported in 28% of DS subjects by 30 years of age, as well as changes in personality and behavior (Head, 2012). Early Aβ deposition may be related to subtle declines in episodic and/or executive functioning, called mild cognitive impairment (Hartley, 2017). A recent study using positron emission tomography tracer [11C] Pittsburgh compound B (PiB) to measure brain amyloid burden in DS subjects has shown that an increase of global amyloid-β was related to decline in verbal episodic memory, visual episodic memory, executive functioning, and fine motor processing speed. DS subjects who were consistently PiB+ demonstrated worsening of episodic memory, whereas those who were consistently PiB− evidenced stable or improved performance (Hartley, 2017). The diagnosis of cognitive decline can be difficult in the DS population since it can appear similar to symptoms of intellectual disability, so improved diagnostic methods are being investigated. Compounding the difficulty in diagnosis is that early symptoms are not uniformly exhibited. For example, memory loss is a key early clinical symptom of developing dementia, but this does not hold true in the DS population.

Current treatment for cognitive decline in DS is very limited, with the majority of research focused on dementia or AD. Therapies that have been investigated and shown promise for these indications, such as cholinesterase inhibitors, have so far shown to have poor efficacy in DS subjects experiencing cognitive decline (Prasher, 2002). In contrast to AD, immunotherapies targeting Aβ are not being widely addressed in DS.

WO2013/044147 and Belichenko (2016) describe vaccination of Ts65Dn mice, a model of DS, with a vaccine containing the Aβ 1-15 peptide embedded into liposomes.

### DESCRIPTION OF THE INVENTION

The present invention arises from clinical trials of the ACI-24 vaccine comprising an anti-abeta (anti-Aβ) antigen (comprising amino acids 1-15 of the human Aβ sequence) and MPLA adjuvant in a liposomal formulation. The vaccine was able to induce anti-abeta antibody titers in human subjects with AD (mild to moderate AD) at the two highest doses tested (300 and 1000 μg of antigen) without inducing serious adverse event (SAE) related to the study treatment (investigational product). More specifically, the vaccine was able to induce anti-abeta antibody titers in human subjects with AD (mild to moderate AD) when administered at 300 and 1000 μg of antigen along with the following clinical observations:
Safety was considered good in the study at all doses tested;
No SAE related to study treatment was observed;
No signal of CNS inflammation or other important unwanted reactions to the vaccine;
No ARIA-E and ARIA-H observed (1 tiny lesion with low signal on hemosequence suspicious for a microbleed observed at 100 μg dose of ACI-24 (possible artefact) in one AD patient);
No indication of the development of meningoencephalitis;
No observed T-cell activation and induction of inflammatory cytokines.

Similarly, the vaccine was able to induce anti-abeta antibody titers in human subjects with DS at both doses tested (300 and 1000 μg of antigen) without inducing serious adverse event (SAE) related to the study treatment (investigational product). More specifically, the vaccine was able to induce anti-abeta antibody titers in human subjects with DS when administered at 300 and 1000 μg of antigen, with an early onset response (first increase in titers observed at 4 weeks) and a boosting effect over time (as measured by Meso Scale Discovery (MSD) immunoassay), along with the following clinical observations:
Safety was considered good in the study at all doses tested so far;
No SAE reported;
No signal of CNS inflammation or other important unwanted reactions to the vaccine;
No ARIA-E and ARIA-H observed;
No indication of the development of meningoencephalitis;
No T-cell activation and induction of inflammatory cytokines observed so far.

Accordingly, the invention provides a method of inducing an anti-Aβ immune response in a human subject without inducing a serious adverse event (i.e. a SAE caused by the treatment), the method comprising administering to the human subject a liposomal vaccine composition comprising:
a. A β-amyloid (Aβ)-derived peptide antigen displayed on the surface of the liposome that comprises, consists essentially of or consists of amino acids 1-15 of Aβ
b. An adjuvant comprising monophosphoryl lipid A (MPLA)
   wherein the β-amyloid (Aβ)-derived peptide antigen is administered in an amount of 300-2000 μg.

Such methods may also be expressed in the form of a medical use. Accordingly, the invention also provides a liposomal vaccine composition comprising:
a. A β-amyloid (Aβ)-derived peptide antigen displayed on the surface of the liposome that comprises, consists essentially of or consists of amino acids 1-15 of Aβ
b. An adjuvant comprising monophosphoryl lipid A (MPLA)
   for use in inducing an anti-Aβ immune response in a human subject without inducing a serious adverse event (i.e. a SAE caused by the treatment), wherein the β-amyloid (Aβ)-derived peptide antigen is administered in an amount of 300-2000 μg.

Similarly, the invention provides for use of a liposomal vaccine composition comprising:
a. A β-amyloid (Aβ)-derived peptide antigen displayed on the surface of the liposome that comprises, consists essentially of or consists of amino acids 1-15 of Aβ
b. An adjuvant comprising monophosphoryl lipid A (MPLA)
   in the manufacture of a medicament for use in inducing an anti-Aβ immune response in a human subject without inducing a serious adverse event (i.e. a SAE caused by the treatment), wherein the β-amyloid (Aβ)-derived peptide antigen is administered in an amount of 300-2000 μg.

All embodiments herein apply to such methods or medical uses, however expressed.

As introduced above and described in further detail herein, it has been demonstrated that the liposomal compositions of the invention are safe for administration to human subjects. The compositions are safe when administered at dosages that generate a beneficial anti-Aβ immune response. Safety is measured with reference to the absence of any serious adverse event caused by administration of the liposomal vaccine composition. “Serious adverse event”, or “SAE”, may be defined as any adverse event or adverse reaction that results in death, is life-threatening, requires hospitalisation or prolongation of existing hospitalisation, results in persistent or significant disability or incapacity, or is a congenital anomaly or birth defect. “Life-threatening” in the definition of a serious adverse event refers to an event in which the subject was at risk of death at the time of the event. It does not refer to an event which hypothetically might have caused death if it were more severe. Important adverse events/reactions that are not immediately life-threatening or do not result in death or hospitalisation, but may jeopardise the subject or may require intervention to prevent one of the other outcomes listed in the definition above, should also be considered serious. Although interpretation of such events requires medical judgement, the investigators participating in human clinical trials are able to determine whether a serious adverse event has occurred during the clinical trial and whether or not this is related to the administration of the liposomal vaccine composition. For the avoidance of doubt, it is possible that a serious adverse event may occur in a given subject which is not related to (induced or caused by) administration of the liposomal vaccine composition. This is not precluded by the invention.

Specific SAEs which are not induced when the liposomal compositions of the invention are administered include:
CNS inflammation or other important unwanted reactions to the vaccine;
ARIA-E and ARIA-H;
Meningoencephalitis;
T-cell activation and induction of inflammatory cytokines.

By “T-cell activation” in the context of the liposomal compositions of the invention is meant Aβ-specific T-cell activation. As discussed above, in a previous study (Orgogozo, 2003) some patients developed an inflammatory reaction considered to be due to a T-cell-mediated response against full length Aβ1-42. This T-cell-mediated response against full length Aβ1-42 is avoided using the liposomal compositions of the invention, which are based on Aβ1-15. Aβ-specific T-cell activation can be evaluated using enzyme-linked immune absorbent spot (ELISpot), which is a type of assay that focuses on quantitatively measuring the frequency of cytokine secretion for a single cell.

Amyloid-related imaging abnormalities (ARIA) are abnormal signals seen in neuroimaging of Alzheimer's Disease patients, associated with amyloid-modifying therapies. ARIA-E refers to cerebral edema, involving the breakdown of the tight endothelial junctions of the blood-brain barrier and subsequent accumulation of fluid. ARIA-H refers to cerebral microhaemorrhages (mH), small haemorrhages in the brain, often accompanied by hemosiderosis.

SAEs may be absent during the period over which the liposomal vaccine composition is administered. SAEs may be absent for a suitable period of time following the final administration of the liposomal vaccine composition. For example, there may be no SAEs after 12, 24, 36 or 48 weeks, or 1, 2 or 3 years following the final administration of the liposomal vaccine composition.

As presented herein, and unless otherwise specified, dosage amounts relate to the per dose administration amount of the β-amyloid (Aβ)-derived peptide antigen in the liposomal vaccine composition. Thus, as in ACI-24, the dosages are, unless otherwise specified, expressed with reference to tetrapalmitoylated Abeta 1-15 as described herein and also in SEQ ID NO: 1:

SEQ ID NO: 1-Tetrapalmitoylated Abeta 1-15
H-Lys(palmitoyl)-Lys(palmitoyl)-Asp-Ala-Glu-Phe-
Arg-His-Asp-Ser-Gly-Tyr-Glu-Val-His-His-Gln-Lys
(palmitoyl)-Lys(palmitoyl)-OH

Where particular values are specified, these values are subject to manufacturing tolerances as would be appreciated by one skilled in the art. Typically, the specified dose covers 15% variation either side of the indicated value. For example, a specified dose of 1000 μg of β-amyloid (Aβ)-derived peptide antigen encompasses from 850 to 1150 μg of β-amyloid (Aβ)-derived peptide antigen. Liposomal vaccine compositions as described herein were safe when the β-amyloid (Aβ)-derived peptide antigen was administered in an amount of 10-1000 μg. However, doses of at least 300 μg were required in order to generate an anti-Aβ immune response. The two highest administered doses (300 μg and 1000 μg) resulted in a measurable anti-Aβ immune response. The response was potentially dose-dependent. The term “anti-Aβ immune response” refers to the production of anti-Aβ antibodies that bind to Aβ by the human subject in response to administration of the liposomal vaccine composition. The response may thus also be referred to as an anti-Aβ antibody response. The antibodies may comprise antibodies of IgM isotype. The antibodies preferably comprise antibodies of IgG isotype. The antibody response is typically polyclonal. This response can be measured in suitable samples taken from the human subject, such as a serum-containing sample. Thus, the sample may comprise, or be derived from, a blood sample. The antibodies preferably bind to pathological forms of Aβ, defined as forms of Aβ that comprise β-sheet multimers. The antibodies produced may therefore be termed “Aβ-specific” antibodies. The anti-Aβ immune response may be measured by any suitable method, such as an ELISA. For example, the anti-Aβ immune response may be measured by a method in which Aβ, such as Aβ1-42, is coated on a solid support to which is applied the sample from the human subject. A secondary antibody may be used to detect binding of antibodies from the sample to the immobilized Aβ. Such methods may be quantitative. The secondary antibody may be an anti-Ig antibody, thereby permitting all isotypes to be detected. The secondary antibody may be an anti-IgG antibody. This may permit Aβ-specific IgG titers to be measured.

Thus, according to all aspects of the invention, the β-amyloid (Aβ)-derived peptide antigen (dosage expressed for tetrapalmitoylated Abeta 1-15 as set forth in SEQ ID NO: 1) is administered in an amount of 300-2000 μg. This dosage combines safety (no induced SAE) with the ability to generate an anti-Aβ immune response. Since the anti-Aβ immune response was increased, and safety retained, at higher tested doses, higher dosages within this range may be advantageous. For example, according to some embodiments, the β-amyloid (Aβ)-derived peptide antigen is administered in an amount of 500-2000 μg, preferably 1000-1500 μg. In certain embodiments, the β-amyloid (Aβ)-derived peptide antigen (dosage expressed for tetrapalmitoylated Abeta 1-15 as set forth in SEQ ID NO: 1) is administered in an amount of 1000 μg. In preferred embodiments, the β-amyloid (Aβ)-derived peptide antigen of SEQ ID NO: 1 (tetrapalmitoylated Abeta 1-15) is administered in an amount of 300-2000 μg.

As would be readily appreciated by one skilled in the art, dosages may alternatively be expressed with reference to the equivalent amount of Abeta 1-15 alone (i.e. without lysine residues and palmitoylation) as described herein and also in SEQ ID NO: 2:

SEQ ID NO: 2-Abeta 1-15
H-Asp-Ala-Glu-Phe-Arg-His-Asp-Ser-Gly-Tyr-Glu-Val-
His-His-Gln-OH

Thus, according to some aspects of the invention, the β-amyloid (Aβ)-derived peptide antigen (dosage expressed for Abeta 1-15 as set forth in SEQ ID NO: 2) is administered in an amount of 152-1016 μg (equivalent to 300-2000 μg tetrapalmitoylated Abeta 1-15 as set forth in SEQ ID NO: 1). This dosage combines safety (no induced SAE) with the ability to generate an anti-Aβ immune response. Since the anti-Aβ immune response was increased, and safety retained, at higher tested doses, higher dosages within this range may be advantageous. For example, according to some embodiments, the β-amyloid (Aβ)-derived peptide antigen (dosage expressed for Abeta 1-15 as set forth in SEQ ID NO: 2) is administered in an amount of 255-1016 μg, preferably 510-767 μg. In certain embodiments, the β-amyloid (Aβ)-derived peptide antigen (dosage expressed for Abeta 1-15 as set forth in SEQ ID NO: 2) is administered in an amount between 130 and 177 μg, preferably 152 μg. In certain embodiments, the β-amyloid (Aβ)-derived peptide antigen (dosage expressed for Abeta 1-15 as set forth in SEQ ID NO: 2) is administered in an amount of 432-588 μg, preferably 510 μg. In certain embodiments, the β-amyloid (Aβ)-derived peptide antigen (dosage expressed for Abeta 1-15 as set forth in SEQ ID NO: 2) is administered in an amount of 510 μg. In certain embodiments, the β-amyloid (Aβ)-derived peptide antigen of SEQ ID NO: 2 is administered in an amount of 152-1016 μg.

Additional beneficial effects observed upon administration of the liposomal vaccine compositions of the invention at the specified doses include a dose-dependent reduction in brain amyloid load (as measured by PET, see FIG. 1), an improvement in cognition as measured by Mini Mental State Examination (MMSE) during the treatment period (FIG. 2) and an improvement in cognition/function as measured by CDR-SB during the treatment period (FIG. 3). The Mini Mental State Examination (MMSE) (Folstein 1975) is well-known in the field; it is the most commonly used test for complaints of problems with memory or other mental abilities and is used by clinicians to help detect cognitive impairment and to help assess its progression and severity. It consists of a series of questions and tests, each of which scores points if answered correctly. The MMSE tests a number of different mental abilities, including a person's memory, attention and language. The score is from 0 to 30 with 30 being the best possible and 0 being the worst possible score. As FIG. 2 shows, there was an improvement in MMSE during the treatment period when the β-amyloid (Aβ)-derived peptide antigen was administered in an amount of 1000 μg. It must be noted that the study was not powered on this particular parameter.

The Clinical Dementia Rating scale or CDR scale is a numeric scale used to quantify the severity of symptoms of AD (i.e. its ‘stage’). The system was developed at Washington University School of Medicine (Hughes et al 1982) and involves a qualified health professional assessing the human subject's cognitive and functional performance in six areas via a semi-structure interview: memory, orientation, judgment and problem solving, community affairs, home and hobbies, and personal care. Scores in each of these may be combined to obtain a composite score ranging from 0 (no symptoms) to 3 (severe), referred to as the sum of boxes (CDR-SB). The CDR-SB score may therefore range from 0 to 18 points. As FIG. 3 shows, there was a relative improvement in CDR-SB during the treatment period when the β-amyloid (Aβ)-derived peptide antigen was administered in an amount of 1000 μg. It must be noted that the study was not powered on this particular parameter.

Additional beneficial effects observed upon administration of the liposomal vaccine compositions of the invention at the specified doses to DS subjects include an early onset response, with an increase in anti-Aβ antibody titers as soon as at 4 weeks, earlier IgG titers as compared to AD patients (per the AD study described in Example 1), a boosting effect observed over time (e.g. as measured by MesoScale Discovery immunoassay), and a consistent response in the majority of patients at the highest dose (e.g. as measured by MesoScale Discovery immunoassay).

The Aβ-derived peptide antigen is displayed on the outer surface of the liposome. This is typically by insertion into the outer surface of the liposome. Insertion into the outer surface of the liposome may be facilitated through attachment of the Aβ-derived peptide antigen to a moiety that inserts into the outer surface of the liposome. The liposome may be any liposome that is suitable to present the Aβ-derived peptide antigen on the surface. Typically, the moiety comprises a hydrophobic moiety to ensure insertion into the lipid bilayer of a liposome. The moiety may be any suitable moiety but is preferably a fatty acid. Thus, in preferred embodiments, the β-amyloid (Aβ)-derived peptide antigen is lipidated. The fatty acid may comprise a palmitoyl residue. The β-amyloid (Aβ)-derived peptide antigen may therefore be palmitoylated. A preferred construction comprises the Aβ-derived peptide antigen (Aβ(1-15)) attached to two palmitoyl residues in the N and C terminal regions of the peptide. Thus, the peptide antigen is tetrapalmitoylated. This may be facilitated by incorporating two amino acids, such as lysine, residues in the N and C terminal regions of the Aβ-derived peptide antigen. The amino acid, such as lysine, residues are palmitoylated.

In some embodiments, the liposome has a negative surface charge; the liposome is anionic. Preferably, the liposome comprises phospholipids and even more preferably, the phospholipids comprise dimyrsitoylphosphatidyl-choline (DMPC) and dimyrsitoylphosphatidyl-glycerol (DMPG). The liposome may further comprise cholesterol. The molar ratios of these three components may be 9:1:7 in some embodiments.

A most preferred construction therefore comprises the Aβ-derived peptide antigen reconstituted in the liposome. Accordingly, these compositions of the invention may generally be referred to herein as “liposomal vaccine compositions of the invention”.

The Aβ-derived peptide antigen induces a B-cell response in the subject. It is a “B-cell antigen”. B-cells are activated to proliferate and produce immunoglobulin (Ig) by cross-linking the B-cell surface Ig receptor. As already explained, Aβ plaques are formed by the 39 to 43 amino acid long Aβ peptide, which is in random coil conformation in its natural non-pathological form. During the transition to the pathological state, it transforms mainly into a β-sheet secondary structure, spontaneously aggregating into insoluble deposits. The Aβ-derived peptide antigen is thus defined herein as a peptide antigen derived from the (maximum of) 43 amino acids of (human) Aβ, but is not full length Aβ. More specifically, the Aβ-derived peptide antigen includes the immunodominant B-cell epitope of Aβ(1-42) but lacks the T-cell epitope found in Aβ(1-42). The Aβ-derived peptide antigen comprises, consists essentially of or consists of 15 contiguous amino acids from the N-terminal 17 amino acids of Aβ. It should be noted that the Aβ-derived peptide antigen may be provided in the context of a larger peptide molecule, the remainder of which is not derived from the Aβ amino acid sequence. For example, the peptide can include additional residues, such as lysine residues to facilitate palmitoylation. Those residues are typically found at the N and C terminus of the peptide. In this context, the term “consists essentially of” means that the Aβ-derived peptide antigen includes the 15 contiguous amino acids from the N-terminal 17 amino acids of Aβ but can include a limited number of additional residues, such as four lysine residues to facilitate palmitoylation. The Aβ-derived peptide antigen comprises, consists essentially of or consists of amino acids 1-15 of Aβ, which may be referred to as “Aβ(1-15)” (WO2007/068411, ACI-24).

The Aβ-derived peptide antigen included in the compositions of the invention adopts a secondary structure that replicates a pathological form of Aβ. Preferably, the Aβ-derived peptide antigen adopts a secondary structure comprising a β-sheet conformation. Even more preferably, the Aβ-derived peptide antigen adopts a predominantly β-sheet conformation when displayed on the surface of the liposome.

The A-derived peptide antigen included in the compositions of the invention is a synthetic peptide. In some embodiments, the Aβ-derived peptide antigen is produced by chemical synthesis.

The liposomal vaccine compositions comprise at least one monophosphoryl lipid A (MPLA) adjuvant. Lipid A based adjuvants derive from lipopolysaccharide (they are chemically modified to reduce toxicity) and have been proven to be safe and effective. The MPLA adjuvant used herein is preferably a synthetic monophosphoryl lipid A (MPLA). As defined herein, the term MPLA encompasses MPLA-derivatives such as Monophosphoryl Hexa-acyl Lipid A, 3-Deacyl (Synthetic) (3D-(6-acyl) PHAD®), PHAD® (Phosphorylated HexaAcyl Disaccharide) and MPL. The MPLA adjuvant may be a Toll-like receptor (TLR) agonist, in particular a TLR4 agonist. The purpose of the adjuvant(s) is to increase or stimulate the immune response in the subject. Preferably, the at least one MPLA adjuvant forms part of a liposome; it may form part of the lipid bilayer. The MPLA adjuvant may be, at least in part, displayed on the outer surface of the liposome; this may be as a consequence of the adjuvant forming part of at least the outer layer of the lipid bilayer. The liposome may effectively function as an adjuvant with the addition of monophosphoryl lipid A (MPLA). The MPLA adjuvant typically forms part of the outer layer of the liposome. The MPLA is typically added during liposomal formation (as explained further herein). Preferred liposomes thus comprise dimyrsitoylphosphatidyl-choline (DMPC), dimyrsitoylphosphatidyl-glycerol (DMPG), cholesterol and MPLA. The molar ratios of these four components may be 9:1:7:0.05 in some embodiments.

In some embodiments of the invention, the compositions of the invention comprise two different adjuvants. Additional adjuvants that may be employed according to the invention include aluminium hydroxide (Alum) and/or CpG amongst others. One or more MPLA adjuvants forming part of a liposome may be combined with an encapsulated adjuvant in some embodiments. In other embodiments, one or more MPLA adjuvants forming part of a liposome may be mixed with a further adjuvant (such as Alum or CpG) when forming the liposomes.

The MPLA adjuvant may be included in the compositions at a dose that correlates with the dose of the β-amyloid (Aβ)-derived peptide antigen. Thus, for example, a liposomal vaccine composition in which the β-amyloid (Aβ)-derived peptide antigen (dosage expressed for tetrapalmitoylated Abeta 1-15 as set forth in SEQ ID NO: 1) is administered in an amount of 1000 μg (which may be between 850 and 1150 μg in view of manufacturing tolerances) may comprise an MPLA adjuvant administered in an amount of 175 μg (which may be between 50 and 300 μg in view of manufacturing tolerances) or in an amount of 225 μg (which may be between 150 and 300 μg in view of manufacturing tolerances). Similarly, a liposomal vaccine composition in which the β-amyloid (Aβ)-derived peptide antigen (dosage expressed for tetrapalmitoylated Abeta 1-15 as set forth in SEQ ID NO: 1) is administered in an amount of 300 μg (which may be between 255 and 345 μg in view of manufacturing tolerances) may comprise an MPLA adjuvant administered in an amount of 52.5 μg (which may be between 15 and 90 μg in view of manufacturing tolerances) or in an amount of 67.5 μg (which may be between 45 and 90 μg in view of manufacturing tolerances). The MPLA adjuvant may be administered in an amount of 15-600 μg. This dosage contributes to the safety and efficacy (in terms of the ability to generate an anti-Aβ immune response) of the liposomal vaccine composition. According to some embodiments, the MPLA adjuvant is administered in an amount of 50-600 μg, preferably 150-450 μg. In certain embodiments, the MPLA adjuvant is administered in an amount of 175 μg. As presented herein, where particular values are specified, these values are subject to manufacturing tolerances as would be appreciated by one skilled in the art. Typically, the specified dose of MPLA adjuvant covers around 71% variation either side of the indicated value. In other embodiments, based on development of MPLA stock solutions with a narrower concentration range, the MPLA adjuvant may be administered in an amount of 45-600 μg. This dosage also contributes to the safety and efficacy (in terms of the ability to generate an anti-Aβ immune response) of the liposomal vaccine composition. According to some embodiments, the MPLA adjuvant is administered in an amount of 150-600 μg, preferably 200-450 μg. In certain embodiments, the MPLA adjuvant is administered in an amount of 225 μg. For these embodiments, where particular values are specified, these values are also subject to manufacturing tolerances as would be appreciated by one skilled in the art. Typically, the specified dose of MPLA adjuvant covers around 33% variation either side of the indicated value.

The liposomal vaccine compositions of the invention may be synthesised through known means. See for example WO2005/081872, WO2012/020124, WO2012/055933 and WO2013/044147, each of which is hereby incorporated by reference.

The liposomal vaccine compositions may be administered to the subject by any appropriate route of administration. As the skilled person would be aware, vaccine compositions may be administered by topical, oral, rectal, nasal or parenteral (such as intravenous, intradermal, subcutaneous, or intramuscular) routes. In addition, vaccine compositions may be incorporated into sustained release matrices such as biodegradable polymers, the polymers being implanted in the vicinity of, or in close proximity to, where delivery is desired. However, in preferred embodiments, the vaccine composition is administered by injection, most preferably intramuscularly or subcutaneously. Typical volumes of the injectable dosage forms of the liposomal vaccine compositions are between 0.01 to 10 ml, such as 0.75 to 2.5 ml, preferably around 2.5 ml.

The liposomal vaccine compositions may be administered a single time to the subject to generate a protective immune response. However, generally, the liposomal vaccine compositions are administered multiple times to the same subject. Thus, so-called prime-boost regimens may be employed according to the invention. Administration of the vaccine is typically separated by an intervening period of at least 1 week and often around 1-12 months. Safety and efficacy (in terms of the ability to generate an anti-Aβ immune response) has been confirmed for the liposomal vaccine compositions when administered regularly over a long period of time. In some embodiments, the liposomal vaccine composition is administered at a first time and is administered at a second time 1 to 4 weeks later. The liposomal vaccine composition may be administered 2, 3, 4, 5, 6, 7, 8, 9, 10 or more times provided a suitable period of time is allowed between administrations. The liposomal vaccine composition may be administered 2, 3, 4, 5, 6, 7, 8, 9, 10, 11 or 12 times over the course of a 12 month period provided a suitable period of time is allowed between administrations. The liposomal vaccine composition may be administered indefinitely provided a suitable period of time is allowed between administrations. A suitable period of time is typically at least 1 week and often around 1-12 months. The period of time may be based on monitoring of the individual subject. Monitoring may comprise monitoring the disease status of the subject and/or monitoring levels of immune response of the subject over time. Tests (e.g. MMSE, amyloid PET-scan or anti-Aβ immune response) are described herein that allow the course of disease to be followed. In prophylactic applications, the liposomal vaccine compositions may be administered less frequently compared to therapeutic methods, and may be administered according to a regular schedule. Monitoring may be employed in the context of prophylactic methods. For example, in subjects with a predisposition to developing an amyloid-beta associated disease or condition or a condition characterised by, or associated with, loss of cognitive memory capacity. Suitable tests and biomarkers are described herein and include monitoring brain Abeta levels using amyloid PET-scan (which may be absent in early prevention), monitoring AD progression biomarkers such as Tau, phosphorylated Tau and Abeta levels (Aβ1-42 and Aβ1-40) in blood and/or CSF, Neurofilament light Chain in blood and/or CSF, measuring efficacy on clinical/cognitive parameters and measuring immune response in serum and/or CSF including, but not limited to anti-Abeta1-42 IgM titers and/or anti-Abeta1-42 IgG titers in blood.

Where time periods for a vaccination regimen are described herein, the initial administration of the liposomal vaccine composition is considered time zero (0). In some embodiments, the liposomal vaccine composition is administered every 4-12 weeks for a period of at least 48 weeks. For example, the liposomal vaccine composition may be administered every 4 weeks for a period of 12 weeks and every 12 weeks for a further period of at least 36 weeks. This would thus include 4 separate administrations of the liposomal vaccine composition at weeks 0, 4, 8 and 12, followed by 3 separate administrations of the liposomal vaccine composition at weeks 24, 36 and 48. According to all administration regimes, the liposomal vaccine composition may be additionally administered as required at a later time point. Typically this is after the completion of the initial administration schedule (“the schedule”). It may thus be referred to as a “booster” administration. Such a further administration may occur at a suitable time point after completion of the initial administration schedule; such as 4, 12, 24, 26, 36, or 48 weeks after the final administration according to the schedule or longer such as 1, 2, 2.5, 3, 3.25, 3.5, 4, 5 or more years after the final administration according to the schedule.

As already indicated, the liposomal vaccine compositions induce an anti-Aβ immune response in a human subject without inducing a serious adverse event. The liposomal vaccine compositions may be administered to human subjects in order to treat, prevent, induce a protective immune response against or alleviate the symptoms associated with an amyloid-beta associated disease or condition or a condition characterised by, or associated with, loss of cognitive memory capacity. The liposomal vaccine compositions may thus be administered for both prophylactic and therapeutic purposes in human subjects.

The amyloid-beta associated disease or condition may be a neurological disorder such as (and in particular) Alzheimer's Disease (AD). Other examples of amyloid-beta associated diseases or conditions according to the invention include mild cognitive impairment (MCI), Down syndrome (DS), including Down syndrome-related Alzheimer's disease, cardiac amyloidosis, cerebral amyloid angiopathy (CAA), multiple sclerosis, Parkinson's disease, Lewy body dementia, ALS (amyotrophic lateral sclerosis), Adult Onset Diabetes, inclusion body myositis (IBM), ocular amyloidosis, glaucoma, macular degeneration, lattice dystrophy and optic neuritis. Many of these conditions are characterized by, or associated with, loss of cognitive memory capacity. Conditions characterized by, or associated with, loss of cognitive memory capacity according to the invention therefore include AD, mild cognitive impairment (MCI), Down syndrome, including Down syndrome-related Alzheimer's disease, cardiac amyloidosis, cerebral amyloid angiopathy (CAA), multiple sclerosis, Parkinson's disease, Lewy body dementia, ALS (amyotrophic lateral sclerosis) and inclusion body myositis (IBM).

Thus, the invention is directed to treatment and prevention of an amyloid-beta associated disease or condition or a condition characterized by, or associated with, loss of cognitive memory capacity, comprising administering the vaccine of the invention. The amyloid-beta associated disease or condition or a condition characterized by, or associated with, loss of cognitive memory capacity, includes Alzheimer's Disease, mild cognitive impairment (MCI), Down syndrome (DS), including Down syndrome-related Alzheimer's disease, cardiac amyloidosis, cerebral amyloid angiopathy (CAA), multiple sclerosis, Parkinson's disease, Lewy body dementia, ALS (amyotrophic lateral sclerosis), Adult Onset Diabetes, inclusion body myositis (IBM), ocular amyloidosis, glaucoma, macular degeneration, lattice dystrophy and optic neuritis, preferably Alzheimer's disease (AD), Down syndrome (DS) and Down syndrome-related Alzheimer's disease.

For AD, it has been observed that intervention may be most effective as early as possible in the development of cognitive impairment. Thus, prophylactic administration may be advantageous, particularly in the presence of other risk factors. In such embodiments, the human subject, prior to treatment, may display an absence of cognitive impairment consistent with a Mini Mental State Examination (MMSE) score of around 30. For the avoidance of doubt, this score indicates no cognitive impairment.

In addition, administration to human subjects with early AD may also be beneficial. In some embodiments, the human subject, prior to treatment, displays cognitive impairment consistent with a Mini Mental State Examination (MMSE) score of at least 18 (so 18-30), such as 18-28, preferably at least 20 (so 20-30), such as 20-28. In some embodiments, the human subject is suffering from AD, in particular early AD. Such subjects may display cognitive impairment consistent with a MMSE score of at least 20. Early AD includes mild cognitive impairment due to AD and mild AD. In some embodiments, the human subject is suffering from mild AD. Such subjects may display cognitive impairment consistent with a MMSE score of 20-28. In other embodiments, the subject is not suffering from severe (late stage) AD. In further embodiments, the human subject is suffering from early AD, mild AD, mild to moderate AD or moderate AD. Such subjects may display cognitive impairment consistent with a MMSE score of at least 12.

In specific embodiments, the human subject is suffering from mild to moderate AD. Such subjects may display cognitive impairment consistent with a MMSE score of at 12-28. In specific embodiments, the human subject is suffering from moderate AD. Such subjects may display cognitive impairment consistent with a MMSE score of 12-19.

Other factors that may be included when selecting subjects for treatment include age. For example, the subject may be over 40 years of age.

As already discussed, a key feature of adult subjects with DS is their increased risk of developing similar clinical symptoms of Alzheimer's Disease (AD), characterized by a decline in specific cognitive domains suggestive of a diagnosis of dementia in the most advanced stage. Virtually all subjects with DS older than 40 years exhibit neuropathological changes similar to AD, in the form of senile plaque formation and neurofibrillary tangles (Head, 2012). Thus, when reference is made herein to treating, preventing, inducing a protective immune response against or alleviate the symptoms associated with DS specifically, it is intended to relate to AD-like symptoms in DS subjects. Preventive treatment may be applied to those subjects without evidence of beta amyloid plaque formation and neurofibrillary tangles. As already discussed, a study using positron emission tomography tracer [11C] Pittsburgh compound B (PiB) to measure brain amyloid burden in DS subjects has shown that an increase of global amyloid-s was related to decline in verbal episodic memory, visual episodic memory, executive functioning, and fine motor processing speed. DS subjects who were consistently PiB+ demonstrated worsening of episodic memory, whereas those who were consistently PiB− evidenced stable or improved performance (Hartley, 2017). Thus, preventive treatment may be applied to those subjects who are PiB−. Conversely, therapeutic treatment may be applied to those subjects with evidence of beta amyloid plaque formation and neurofibrillary tangles and/or who are PiB+. DS is a population at increased risk for AD-like disease. It offers opportunities for exploring effective treatments for AD that will benefit both the DS and general populations. Homogeneity in pathogenesis, age-related disease onset and absence of other dementias powerfully enable prevention trials of AD-like symptoms in DS. A focus in DS subjects is prevention therapy. Biomarker endpoints of Alzheimer pathology may be adopted to monitor the therapy. Examples include Abeta levels, total tau, phosphorylated Tau protein, soluble amyloid precursor protein alpha (sAPPα), soluble amyloid precursor protein beta (sAPPβ), Orexin-A, Neurofilament light chain (NfL), inflammatory cytokines, angiogenic proteins and vascular injury markers in plasma and/or in CSF, TLR-4 expression may be adopted to monitor the therapy. PET-scan imaging may also be employed, such as using positron emission tomography tracer [11C] Pittsburgh compound B (PiB), Florbetapir or florbetaben, to measure brain amyloid burden in DS subjects (Hartley, 2017), and potentially Tau positron emission tomography tracers such as flortaucipir or PI-2620. Free, total and complexed IgG titers may be measured. Free, total and complexed IgM titers may be measured. Clinical efficacy may be measured notably by using Clinical Global Impression of Change (CGIC) and/or by cognition tests (e.g., Cambridge Neuropsychological Test Automated Battery (CANTAB) motor control, reaction time, paired associative learning, Cued Recall Test (CRT), Cambridge Cognitive Examination—Down Syndrome (CAMCOG-DS), modified Selective Reminding Test (SRT), NEuroPSYchological Assessment-II—Train and Car Subtest (NEPSY-II), Kaufman Brief Intelligence Test 2 (KBIT-2)); Brief Praxis Test (BPT4), behavior (e.g. by Vineland Adaptive Behavior Scale (VABS), Neuropsychiatric Inventory (NPI) and by assessing the progression to dementia (eg., Dementia Screening Questionnaire for Individuals with Intellectual Disabilities (DSQIID)).

In human subjects with DS, assessment by MMSE may not be appropriate. Similarly, the age considerations may be different (e.g. due to shorter life expectancy). Male or female subjects with DS may be treated at any age, in particular prophylactically. As already mentioned preventive treatments may be applied to subjects without evidence of beta amyloid plaque formation and neurofibrillary tangles. Conversely, therapeutic treatment may be applied to those subjects with evidence of beta amyloid plaque formation and neurofibrillary tangles. Human subjects with DS may be in the pre-clinical stage of AD, with no amyloid-related cognitive decline. The treated subjects may be 50 years old or less, such as 45, 40, 35, 30 or 25 years or less. Human subjects with DS amenable to treatment may be identified as having mild to moderate intellectual disability using the Diagnostic and Statistical Manual of Mental Disorders (DSM-5) classification. DSM-5 is the 2013 update to the Diagnostic and Statistical Manual of Mental Disorders, the taxonomic and diagnostic tool published by the American Psychiatric Association (APA). In the United States, the DSM serves as the principal authority for psychiatric diagnoses.

Human subjects amenable to treatment may be identified as PET-scan positive for Aβ deposits according to some embodiments. Such Aβ deposits are found in patients with early AD (mild cognitive impairment due to AD and mild AD) and also in more advanced stages of AD, such as moderate AD. For example florbetaben positron emission tomography (PET) may be employed to investigate amyloid load in the brain.

Human subjects amenable to treatment may be identified on the basis of CDR score, which may be a CDR-SB score as introduced above. A CDR-SB score of 0 may identify the subject as normal. Such subjects may be amenable to prophylactic treatment, potentially in the presence of other risk factors. A CDR-SB score of 0.5-2.5 may identify a subject with MCI. A CDR-SB score of 2.5-4.0 may identify a subject with very mild AD. A CDR-SB score of 4.5-9.0 may identify a subject with mild AD. A CDR-SB score of 9.5-15.5 may identify a subject with moderate AD. A CDR-SB score of 16.0-18.0 may identify a subject with severe AD. See O'Bryant et al., Arch Neurol. 2010; 67(6):746-749. doi:10.1001/archneurol.2010.115. As already mentioned, administration to human subjects with early stage disease (cognitive impairment or AD) may also be beneficial. Thus, in some embodiments, the human subject, prior to treatment, displays cognitive impairment consistent with a CDR-SB score of no more than 15.5 such as 0.5-15.5, or no more than 9.0, such as 0.5-9.0.

Human subjects amenable to treatment may be identified on the basis of the Montreal Cognitive Assessment (MoCA), which is a 30-question test that takes around 10 to 12 minutes to complete (Nasreddine Z S, Phillips N A, et al. The Montreal Cognitive Assessment, MoCA: A brief screening tool for mild cognitive impairment. J Am Geriatr Soc. 2005; 53:695-699). The MoCA evaluates different types of cognitive abilities. These include orientation, short-term memory/delayed recall, executive function/visuospatial ability, language abilities; abstraction, animal naming, attention and a clock-drawing test. Scores on the MoCA range from zero to 30, with a score of 26 and higher generally considered normal. In the initial study data establishing the MoCA, normal controls had an average score of 27.4, compared with 22.1 in people with mild cognitive impairment (MCI) and 16.2 in subjects with Alzheimer's disease. Thus, a MoCA score less than 26 may identify a subject as amenable to therapeutic treatment. A score of 26 or higher may identify a subject as amenable to prophylactic treatment, potentially in the presence of other risk factors. As already mentioned, administration to human subjects with early stage disease may also be beneficial. Thus, in some embodiments, the human subject, prior to treatment, displays cognitive impairment consistent with a MoCA score of 16-26.

### DESCRIPTION OF THE FIGURES

FIG. 1. Abeta florbetaben Positron emission tomography (PET) exploratory analysis showed a dose dependent trend in reduction of accumulation of brain amyloid observed in cohorts 3 and 4 at week 52. PET scans not conducted for Cohort 1. SUVR-MCG stands for Standardised Uptake Value Ratio-Mean Cerebellar Gray.

FIG. 2. Change in Mini-mental state examination (MMSE) Total Score indicates a positive trend on cognition measured by MMSE observed during the treatment period for the highest dose versus placebo and lower doses.

FIG. 3. Change in Clinical Dementia Rating scale—Sum of Boxes (CDR-SB) score indicates a positive trend on cognition/function measured by CDR-SB observed during the treatment period for the highest doses versus placebo and lower doses.

Table of abbreviations

| | |
|---|---|
| AD | Alzheimer's Disease |
| ARIA-E | Amyloid-related imaging abnormalities-vasogenic edema |
| ARIA-H | Amyloid-related imaging abnormalities-microhemorrhages, |
| | superficial siderosis |
| Aβ | Amyloid beta (abeta) |
| BPT | Brief Praxis Test |
| CANTAB | Cambridge Neuropsychological Test Automated Battery |
| CDR | Clinical Dementia Rating scale |
| CDR-SB | Clinical Dementia Rating scale-Sum of Boxes |
| CGIC | Clinical Global Impression of Change |
| CNS | Central Nervous System |
| CSF | Cerebrospinal Fluid |
| DMPC | 1,2-Dimyristoyl-sn-glycero-3-phosphocholine |
| DMPG | 1,2-Dimyristoyl-sn-glycero-3-phosphorylglycerol |
| DSMB | Data and Safety Monitoring Board |
| ECG | Electrocardiogram |
| ELISA | Enzyme-linked immunosorbent assay |
| ELISPOT | Enzyme-linked immune absorbent spot |
| GABA | Gamma-Aminobutyric acid |
| GLP | Good Laboratory Practice |
| HE | Hematoxylin and eosin |
| IFN | Interferon |
| IL | interleukin |
| i.m. | intramuscular |
| Ig | Immunoglobulin |
| MMSE | Mini-Mental State Examination |
| MPLA | Monophosphoryl Lipid A |
| MRI | Magnetic Resonance Imaging |
| NIA-AA | National Institute on Aging-Alzheimer's Association |
| NINCDS- | National Institute of Neurological and Communicative |
| ADRDA | Diseases and Stroke-Alzheimer's Disease and |
| | Related Disorders Association |
| NOAEL | No Observed Adverse Effet Level |
| NPI | Neuropsychiatric Inventory |
| Pal1-15 | Tetrapalmitoylated Aβ1-15 |
| PBS | Phosphate buffered saline |
| PET | Positron Emission Tomography |
| s.c. | Subcutaneous |
| SAE | Serious Adverse Event |
| sAPPα | Soluble amyloid precursor protein alpha |
| sAPPβ | Soluble amyloid precursor protein beta |
| SSRI/SNRI | Selective serotonin reuptake inhibitor/Serotonin- |
| | norepinephrine reuptake inhibitor |
| SGOT | serum glutamic-oxaloacetic transaminase |
| SGPT | Serum glutamic pyruvic transaminase |
| TLR4 | Toll-like receptor 4 |
| TSH | Thyroid-stimulating hormone |
| VABS | Vineland Adaptive Behavior Scale |

The invention will be further understood with reference to the following non-limiting examples:

### Definitions

The MMSE (Folstein 1975) is a widely used test of overall cognitive function, assessing memory, orientation and praxis in a short series of tests. The score is from 0 to 30 with 30 being the best possible and 0 being the worst possible score.

The Clinical Dementia Rating Scale (Hughes et al 1982) is a global rating of the function (it is not only purely functioning since cognition is also being checked with memory) of Alzheimer patients assessed in six categories: memory, orientation, judgement and problem solving, community affairs, home and hobbies and personal care. It is based on a semi-structured interview conducted with the patient and caregiver, by a rater without access to the results of the cognitive tests described above. Each category has scores from 0 (no symptoms) to 3 (severe) and the sum of these items (Sum of Boxes) may therefore range from 0 to 18 points.

Early AD patients include Mild Cognitive Impairment (MCI) due to AD and mild AD.

According to the National Institute on Aging—Alzheimer Association (NIA-AA) criteria, Mild Cognitive Impairment due to Alzheimer's Disease requires evidence of intra-individual decline, manifested by a change in cognition from previously attained levels, as noted by self- or informant report and/or the judgment of a clinician, impaired cognition in at least one domain (but not necessarily episodic memory) relative to age- and education-matched normative values (impairment in more than one cognitive domain is permissible), a preserved independence in functional abilities, no dementia, and a clinical presentation consistent with the phenotype of AD in the absence of other potentially dementing disorders.

Probable AD dementia according to NIA-AA criteria meets criteria for dementia and in addition, has the following main characteristics: insidious onset (symptoms have a gradual onset over months to years, not sudden over hours or days), clear-cut history of worsening of cognition by report or observation; and the initial and most prominent cognitive deficits are evident on history and examination in one of the following categories: Amnestic presentation (it is the most common syndromic presentation of AD dementia. The deficits should include impairment in learning and recall of recently learned information). There should also be evidence of cognitive dysfunction in at least one other cognitive domain); Non-amnestic presentations: Language presentation (the most prominent deficits are in word-finding, but deficits in other cognitive domains should be present); Visuospatial presentation: (the most prominent deficits are in spatial cognition, including object agnosia, impaired face recognition, simultanagnosia, and alexia; deficits in other cognitive domains should be present); Executive dysfunction (the most prominent deficits are impaired reasoning, judgment, and problem solving. Deficits in other cognitive domains should be present).

Early AD patients are patients with the MMSE score of at least 20 (equal or above 20).

They include patients with Mild Cognitive Impairment due to AD and patients with mild AD.

Mild AD patients are patients with the MMSE score of 20 to 28.

Mild-to moderate AD patients are patients with the MMSE score of 12 to 28.

Moderate AD patients are patients with the MMSE score of 12 to 19.

### Example 1. Safety and Efficacy in Humans in Phase I/II AD Trial

### Study Objective:

The overall study objective was to assess the safety, immunogenicity and efficacy of repeated doses of ACI-24 at 4 different dose levels administered to patients with mild to moderate Alzheimer's disease (AD) as diagnosed by the criteria of the National Institute of Neurological and Communicative Diseases and Stroke—Alzheimer's Disease and Related Disorders Association (NINCDS-ADRDA) and with a score at initial screening of 18-28 on the Mini-Mental State Examination (MMSE).

### Primary Objectives:

To assess the safety and tolerability of ACI-24 in patients with mild to moderate Alzheimer's Disease.
To assess the effect of different doses of ACI-24 on induction of anti-Aβ1-42 IgG titer in serum.

### Secondary Objectives:

To explore the efficacy of ACI-24 in reducing Aβ level in the brain of patients with mild to moderate Alzheimer's Disease.
To explore the effect of ACI-24 on T cell activation.
To explore the effects of ACI-24 on putative biomarkers of the progression of Alzheimer's Disease like total tau and phosphorylated tau protein (phosphotau) and Aβ levels (Aβ1-42 and Aβ1-40) in blood and CSF.
To explore the efficacy of ACI-24 on clinical/cognitive endpoints in patients with mild to moderate Alzheimer's Disease.
To explore the induction of immune response in serum and/or CSF including, but not limited to, anti-Aβ1-42 IgM titer in blood.
To explore the induction of inflammatory cytokines in blood.

48 Patients were randomized with a ratio of 3:1 active (ACI-24) versus placebo (normal saline) into 4 dose-cohorts. Patients were administered the study medication 7 times, once every 4 weeks for the first 4 administrations, then once every 12 weeks for the last 3 administrations. The administration schedule of subcutaneous injections was at weeks 0, 4, 8, 12, 24, 36 and 48 with optional booster injections. One additional boosting dose of 300 μg or placebo was administered in 4 patients of cohort 3 (3 were on ACI-24 and 1 was on placebo) who were willing and able to give consent, 6-15 months after the 2 years safety follow-up that is 2.5-3.25 years after the last injection received at visit 16 (V16, week 48 during which the 7th injection was to be administered). An additional boosting dose of ACI-24 1000 μg or placebo was administered to patients of cohort 4, 18 months (week 74) after the first dose. The dose-cohorts were studied sequentially as follows:
Dose-Cohort 1: 10 μg antigen or placebo
Dose-Cohort 2: 100 μg antigen or placebo
Dose-Cohort 3: 300 μg antigen or placebo
Dose-Cohort 4: 1000 μg antigen or placebo

Antigen dose refers to tetrapalmitoylated Aβ1-15 acetate salt. The pharmaceutical form of the vaccine is a suspension for injection (liposomal suspension in PBS). The dose-cohorts were studied in a sequential manner, each cohort having to complete 4 immunizations and safety data including data 2 weeks after the fourth injection (i.e. at visit 8, week 14) being reviewed by the Data and Safety Monitoring Board (DSMB) before the start of enrolment into the next cohort. To further enhance safety an interval of at least one week was planned between first dose administration in the first 4 subjects in each cohort.

### Inclusion Criteria:

Probable AD according to NINCDS-ADRDA criteria.
Florbetaben-PET scan at screening consistent with the presence of amyloid pathology.
Mini-Mental Status Examination (MMSE) 18-28 points*.
Age over 40 and less than 90 years**.
Patients receiving a stable dose of an acetylcholinesterase inhibitor within 4 months prior to baseline.
Patients cared for by a reliable spouse or caregiver to assure compliance, assist with clinical assessments and report safety issues.
Women must be post-menopausal for at least one year, surgically sterilised or using reliable contraceptive measures.
Patient who in the opinion of the investigator are able to understand and provide written informed consent.
Patients and caregivers must be fluent in the language of the study and able to comply with all study procedures.
The patient is lucid and clear and oriented ×4 and is able to provide their written informed consent (applicable only in some countries).
   * For cohort 3 booster injection, the previous lower limit of 18 points for the MMSE was not required but in all cases patients were to be oriented in time, place, awareness person and current activities and able to give informed consent in the opinion of the investigator in order to take part.
   ** For cohort 3 booster injection, no upper age limit applied.

### Exclusion Criteria:

Patients whose MRI scan within the last 6 months shows alternative pathology including severe vascular encephalopathy and/or more than 5 micro-hemorrhages.
Patients with other medical conditions which may influence cognitive performance e.g. Parkinson's disease.
Patients with any unstable medical condition (e.g. epilepsy, uncontrolled hypertension) which would hamper safety assessments.
Patients receiving memantine within 3 months prior to baseline (for cohort 3 booster injection, memantine is allowed).
Patients receiving any anticoagulant drug.
Patients with a history of hemorrhagic stroke.
Patients with a history of non-hemorrhagic stroke or myocardial infarction within the last year.
Patients with a history of major psychiatric disorder within the past 2 years.
Patients with a history of inflammatory neurology disorders including meningoencephalitis.
Clinically significant abnormalities of clinical hematology or biochemistry including, but not limited to, elevations greater than 1.5 times the upper limit of normal of SGOT, SGPT, or creatinine.
Patients with a history of autoimmune disease.
Patients with a history of cancer other than skin cancer within the past 5 years.
Patients who have received any vaccine within the 2 months before baseline.
Patients who have previously received AD immune therapeutic agents or vaccines.
Patients anticipated to receive any vaccination other than flu vaccine during the study.
Patients unable to undergo MRI examination for any reason, including metal implants and claustrophobia.
Patients with a positive HIV test at screening.
Patients with positive syphilis serology.
Women who are pregnant or planning to be pregnant, or who are lactating.

### Results/Conclusions:

48 mild to moderate AD patients were randomized and were exposed to ACI-24 at different dose levels (10 μg, 100 μg, 300 μg and 1000 μg per administration) or placebo with up to seven subcutaneous administrations each, over 12 months. Some patients from the 2 highest dose-cohorts received an additional late booster administration (i.e., a total of 8 subcutaneous injections).

No anti-abeta IgG response was observed in placebo treated patients and in patients treated with the two lowest doses tested (10 and 100 ug of antigen, cohorts 1 and 2). The vaccine was able to induce an anti-abeta antibody response in human subjects in a need thereof at the highest doses tested (300 and 1000 ug of antigen, cohorts 3 and 4) and a dose-dependent anti-Aβ IgG response was observed at the two highest doses. A dose-related late-onset IgG response was observed. Safety was considered good in the study at all doses tested (from 10 μg to 1000 μg of antigen). No SAE related to the study treatment, no signal of CNS Inflammation or other unwanted reactions to the vaccine, no ARIA-E, no ARIA-H (1 tiny lesion with low signal on hemosequence suspicious for a microbleed was noticed at the ACI-24 dose of 100 μg (possible artefact)), no indication of the development of meningoencephalitis and no T-cell activation and induction of inflammatory cytokines were observed.

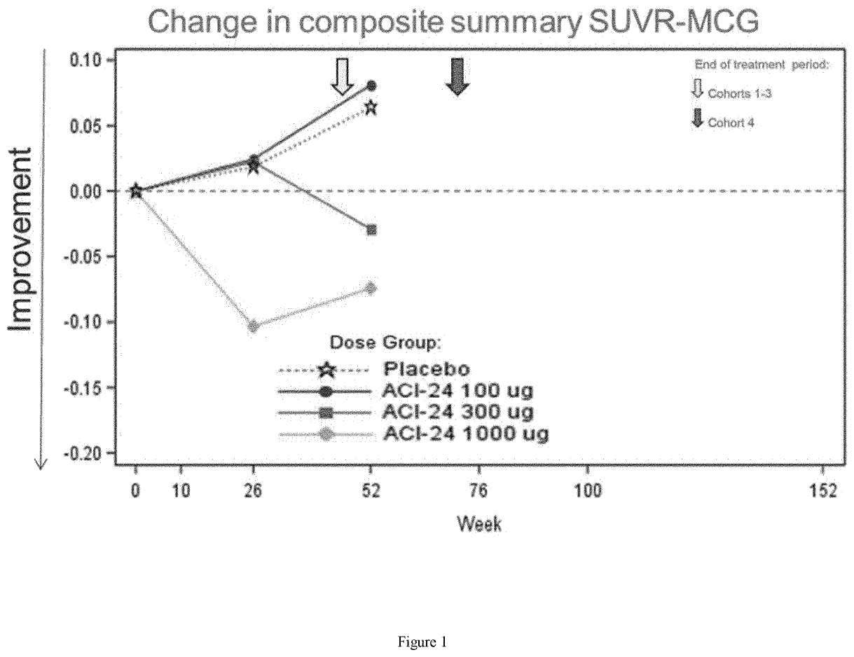

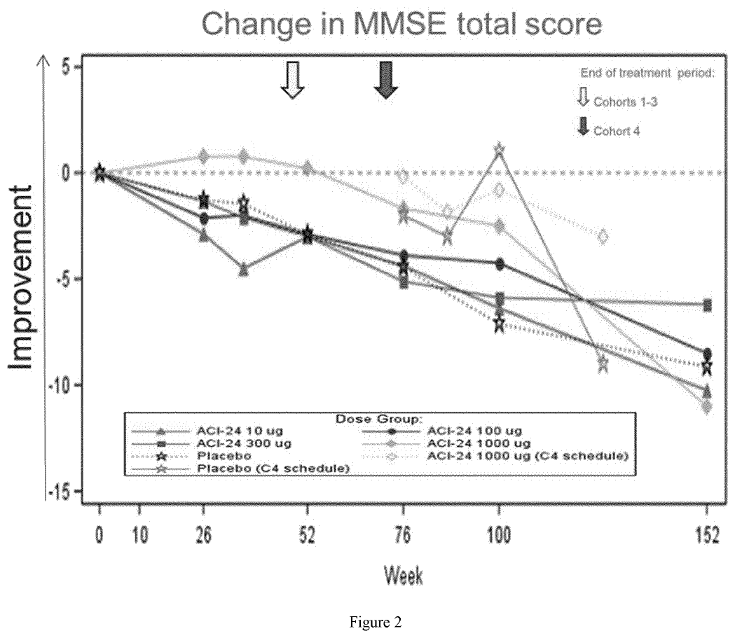

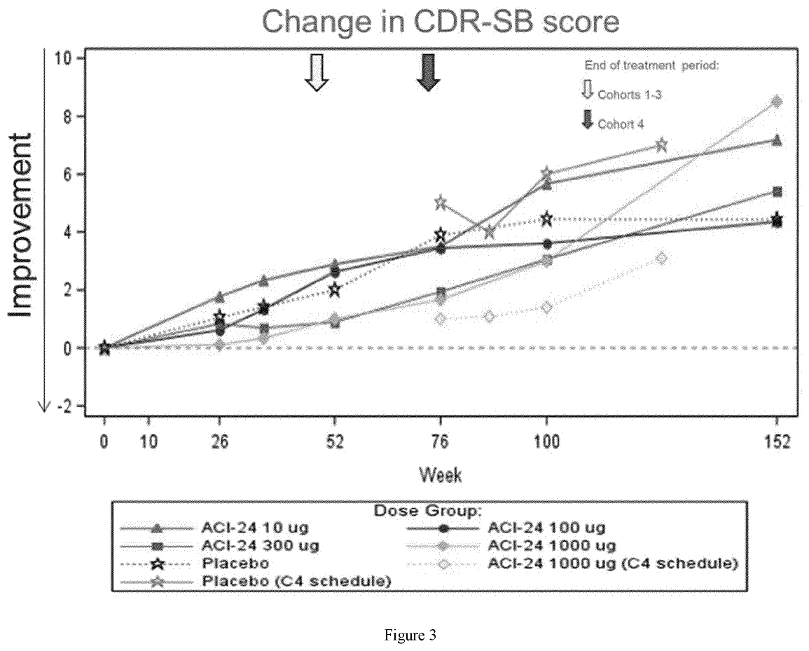

A dose-dependent trend in reduction of brain amyloid accumulation was observed at the two highest doses in both cohorts 3 and 4 at week 52 (FIG. 1). Although the study was not powered on clinical efficacy and PET-scan parameters with a limited number of subjects enrolled (small study population), the exploratory analysis revealed a positive trend on cognition measured by MMSE. This was observed during the treatment period with the highest dose in cohort 4 versus placebo and lower doses (FIG. 2). Similarly, the exploratory analysis revealed a positive trend on cognition/function measured by CDR-SB that was observed during the treatment period for the highest doses versus placebo and lower doses (FIG. 3).

### Example 2. Safety and Efficacy in Humans in Phase II AD Trial

### Study Objective:

The overall study objective is to assess the safety, immunogenicity and efficacy/target engagement of ACI-24 administered to patients with mild Alzheimer's disease (AD) as diagnosed by the criteria of the National Institute on Aging—Alzheimer's Association (NIA-AA) and with a score at initial screening of 20-28 on the Mini-Mental State Examination (MMSE).

### Primary Objectives:

To assess the safety and tolerability of the ACI-24 in patients with mild Alzheimer's disease.
To assess the effects of ACI-24 on induction of anti-Aβ antibody responses in serum.
To assess the effects of ACI-24 on brain amyloid load in patients with mild Alzheimer's disease, assessed by florbetaben-PET imaging at 52 weeks (12 months) and 76 weeks (18 months).

### Secondary Objectives:

To explore the effects of ACI-24 on putative biomarkers of the progression of Alzheimer's disease including concentrations of total tau and phosphorylated tau protein (phosphotau) and Aβ in blood and/or CSF.
To explore the effects of ACI-24 on T cell activation in blood.
To explore the effects of ACI-24 on whole brain and hippocampal volume by volumetric MRI.
To explore the effects of ACI-24 on clinical and cognitive endpoints in patients with mild Alzheimer's disease.
To explore the influence of ACI-24 on blood inflammatory cytokines.

### Inclusion Criteria:

Patients meeting all of the following inclusion criteria at screening should be considered as eligible to participate to the study:

1. Probable AD dementia according to NIA-AA core clinical criteria

2. Florbetaben-PET scan at screening consistent with the presence of amyloid pathology

3. Mini-Mental Status Examination (MMSE) score 20-28 points

4. Age greater than or equal to 50 and less than or equal to 85 years

5. Patients receiving a stable dose of an acetylcholinesterase inhibitor for at least 3 months prior to screening

6. Patients cared for by a reliable spouse or other caregiver to assure compliance, assist with clinical assessments and report safety issues, and spouse or caregiver consents to serve in this role

7. Women must be post-menopausal for at least one year, surgically sterilized or using reliable contraceptive measures

8. Patients who in the opinion of the investigator are able to understand and provide written informed consent

9. Patients and caregivers must be fluent in the official language(s) of the country they are living in and able to comply with all study procedures

10. Patients are lucid and clear and oriented ×4 (awareness of person, knowledge of place, time/date and event) [applicable in some countries only]

In a first cohort, ACI-24 given intramuscularly will be investigated. This study is a multicenter prospective placebo-controlled, double-blind and randomized study to assess treatment with ACI-24 formulations versus placebo over 76 weeks (18 months) in patients with mild Alzheimer's disease. Antigen dose refers to tetrapalmitoylated Aβ1-15 acetate salt. The pharmaceutical form of the vaccine is a suspension for injection (liposomal suspension in PBS).

Cohort 1 with ACI-24:

One dose of ACI-24 at 1000 μg/dose given by the intramuscular route will be tested.

Patients will be randomized with a ratio of 2:1 active (ACI-24) versus placebo.

For patients participating in cohort 1, the treatment period will last 76 weeks with the treatment/placebo being administered 8 times (each time the dose of study treatment will be administered in two separate concomitant intramuscular injections); 4 times with 4 weeks' intervals, 3 times with 12 weeks' intervals and 1 time 26 weeks after the preceding 7th dose. The treatment period is followed by a 24-week period of safety follow-up starting 2 weeks after the last administration. Patients who for some reason receive less than 8 administrations will be followed at least for the same duration after their last administration. Free, total and immune complexed IgG titers will be measured.

### Example 3. Safety and Efficacy in Humans in Phase Ib DS Trial

### Primary Objectives:

To assess the safety and tolerability of ACI-24 in adults with Down Syndrome.
To assess the effect of different doses ACI-24 on induction of anti-Aβ Ig titer in serum.

### Secondary Objectives:

To explore the efficacy of ACI-24 on Clinical Global Impression of Change (CGIC) in adults with Down Syndrome.
To explore the effect of ACI-24 on cognitive (CANTAB motor control, reaction time, paired associative learning; BPT) and behavioral (VABS, NPI) endpoints in adults with Down Syndrome.
To explore the effect of ACI-24 on whole brain, ventricle and hippocampal volume by MRI.
To explore the effect of ACI-24 on peripheral T cell activation.
To explore the effect of ACI-24 on putative biomarkers of Alzheimer pathology in Down Syndrome including Aβ levels, total tau, phosphorylated tau protein (phospho-tau), sAPPα, sAPPβ, Orexin-A, inflammatory cytokines, angiogenic proteins, TLR-4 expression and vascular injury markers in plasma and/or in CSF* (*in subgroup) as applicable.
To assess the effect of different doses ACI-24 on induction of anti-Aβ Ig titer in CSF* (*in subgroup).

### Method:

This is a prospective multi-center, placebo controlled, double-blind and randomized study of 2 doses of ACI-24 treatment versus placebo over 24 months.

The study consists of 2 dose-cohorts of 8 subjects each (6 subjects on ACI-24 300 μg, 6 subjects on ACI-24 1000 μg and 2 subjects on placebo in each dose-cohort) with s.c. injections at month 0, 1, 2, 3, 6, 9 and 12 (or more precisely weeks 0, 4, 8, 12, 24, 36 and 48) with 12 months treatment free safety follow-up. The dose-cohorts are studied sequentially in ascending dose order. The 2nd dose-cohort was started once safety and tolerability data up through visit 8 [week 14] of the last subject of the preceding cohort were reviewed by the Data Safety Monitoring Board (DSMB). Antigen dose refers to tetrapalmitoylated Aβ1-15 acetate salt. The pharmaceutical form of the vaccine is a suspension for injection (liposomal suspension in PBS).

An interim analysis was conducted in this study after visit 8 [week 14] of the last subject of cohort 1 as a basis to allow the dose escalation. The analysis focused on safety and tolerability. The interim analysis was conducted in an unblinded fashion and the unblinded data were presented to the DSMB.

Additional interim analyses are planned to be conducted after visit 9 [week 16], visit 12 [week 28], visit 15 [week 40] and visit 18 [week 52] of the last subject in cohort 1 and in cohort 2 respectively. These analyses focus on safety, tolerability, antibody titer and inflammatory cytokines data (part of biomarkers). Interim analyses at visit 12 [week 28] and visit 18 [week 52] additionally include biomarkers, as well as CGIC, NPI and Vineland data (part of clinical rating scales and cognitive tests).

### Inclusion Criteria:

Males or females with Down Syndrome aged ≥25 to ≤45 years, with a cytogenetic diagnosis being either Trisomy 21 or Complete Unbalanced Translocation of the Chromosome 21.
Subjects and their study partner/legal representative in the opinion of the investigator able to understand and to provide written informed consent.
Written informed consent obtained from subjects and their study partner/legal representative before any trial-related activities.
In the opinion of the investigator able to fully participate in the trial and sufficiently proficient in English to be capable of reliably completing study assessments.
Subjects have a study partner/legal representative who have direct contact with the subjects at least 10 hours per week and who can be asked questions about the subjects.

### Exclusion Criteria:

Subjects weighing less than 40 kg.
IQ less than 40 (as assessed by Kaufman Brief Intelligence Test, Second Edition (KBIT-2).
In the investigators' opinion, any clinically significant current psychiatric or neurologic illness, including a past illness with a risk of recurrence, other than Down syndrome.
Any medical condition likely to significantly hamper the evaluation of safety of the study drug.
DSM-IV criteria for drug or alcohol abuse or dependence currently met within the past five years.
History or presence of uncontrolled seizures. If history of seizures, they must be well controlled with no occurrence of seizures in the past 2 years prior to study screening. The use of antiepileptic medications is permitted.
History of meningitis or meningoencephalitis.
History of malignant neoplasms within 3 years prior to study screening or where there is current evidence of recurrent or metastatic disease.
History of persistent cognitive deficits immediately following head trauma.
History of inflammatory neurology disorders.
History of autoimmune disease with potential for CNS involvement.
MRI scan at screening showing a single area of cerebral vasogenic edema, superficial siderosis, or evidence of a prior macro-hemorrhage, or showing more than four cerebral microhemorrhages (regardless of their anatomical location or diagnostic characterization as “possible” or “definite”).
MRI examination cannot be done for any reason, including metal implants contraindicated for MRI studies and/or severe claustrophobia.
Significant hearing or visual impairment or other issues judged relevant by the investigator preventing to comply with the protocol and to perform the outcome measures.
Severe infections or a major surgical operation within 3 months prior to screening.
History of chronic or recurrent infections judged to be clinically significant by the investigator.
History or presence of immunological or inflammatory conditions which are judged to be clinically significant by the investigator.
Celiac disease not on a gluten free diet for at least 3 months prior to study screening.
Chronic benign skin pathologies, unless viewed as clinically insignificant in the investigator's opinion.
Any vaccine received within the past 2 months before baseline, except influenza vaccine which, if indicated, must be given at least 2 weeks prior to baseline.
Clinically significant arrhythmias or other abnormalities on ECG at screening. (Minor abnormalities documented as clinically insignificant by the investigator will be allowed).
Clinically significant abnormal vital signs including sustained sitting blood pressure greater than 160/90 mmHg.
In the opinion of the site investigator, deviations from normal values for hematologic parameters, liver function tests, and other biochemical measures, that are judged to be clinically significant.
Subjects with treated hypothyroidism not on a stable dose of medication for at least 3 months prior to screening and having clinically significant abnormal serum T-4 and TSH at screening.
Subjects with diabetes mellitus with an HbA1c of ≥8.0%.
Subjects who have been receiving any experimental drug for Down Syndrome with a washout less than 30 days or less than five half-lives of the drug, whichever is longer.
Female subjects being pregnant as confirmed by serum testing at screening or planning to be pregnant or lactating.
Female subjects not using a reliable method of contraception (unless abstaining).
Patient receiving any anticoagulant drug, or aspirin at doses greater than 100 mg daily in the 7 days prior to lumbar puncture (in order to avoid risk of bleeding during scheduled or unscheduled lumbar puncture)
Use of antidepressants other than SSRI/SNRIs at stable dose, antipsychotics (typical or atypical), GABA agonists (e.g. gabapentin), or stimulants (e.g. methylphenidate, modafinil). In exceptional cases, low doses of atypical antipsychotics (e.g. risperidone up to 0.5 mg/day or quetiapine up to 50 mg/day) or benzodiazepines are only allowed after review by the site principal investigator, in consultation with the project director and/or medical monitor.
Current use of immunosuppressant or immunomodulating drugs or their use within the past 6 months prior to study screening. Current use of oral steroids or their use within the past 3 months prior to study screening.
Use of Cholinesterase Inhibitor or use of Glutamatergic drugs (Topiramate, Memantine, Lamotrigine) if not on stable dose for at least 3 months prior to screening.
Subjects who have donated blood or blood products during the 30 days prior to screening who plan to donate blood while participating in the study or within four weeks after completion of the study.

### Results

The trial is a fully enrolled, placebo-controlled, Phase Ib study of the ACI-24 anti-Abeta vaccine. Sixteen subjects have been randomized in the study. The vaccine was able to induce an anti-Abeta antibody response in human subjects in a need thereof at the both doses tested (300 and 1000 ug of antigen). An early-onset IgG response was observed with a first increase in titers at 4 weeks. According to MSD data, a boosting effect could be observed over time, and the anti-Abeta antibody response was consistent in the majority of patients at the highest dose. The vaccine was well tolerated in DS subjects, demonstrating a favourable safety profile at all doses tested. Safety was considered good in the study at both doses tested. There were no subject withdrawals during the treatment period. No SAE related to the study treatment, no signal of CNS inflammation or other important unwanted reactions to the vaccine, no ARIA-E, no ARIA-H, no indication of the development of meningoencephalitis and no T-cell activation and induction of inflammatory cytokines were observed.

The subsequent DS clinical development plan (Example 5) will focus on prevention therapy notably using biomarker endpoints (such as Abeta, Neurofilament, and Tau). The vaccine will be administered at the highest dose (1000 μg) via the intramuscular route to boost immunogenicity further. Two of the selected readouts will be PET-scan imaging and measure of free, total and immune complexed IgG titers generated by the vaccine.

### Example 4. Toxicology Studies

### 4.1 Single Dose Toxicity

Single dose toxicity of ACI-24 was evaluated in two non-clinical models (mice and monkeys). ACI-24 was well tolerated and was not associated with organ toxicity. These two studies are summarized below.

4.1.1 Evaluation of Single-Dose Toxicity following Subcutaneous or Intramuscular Administrations in Mice

### Objective

The potential toxicity, local tolerance and immunogenicity of a single s.c. or i.m. injection of ACI-24 in mice was evaluated.

### Design

The study was conducted under GLP standards. The number of animals, dosage form administered, route of administration and dose-level for each group are summarized in Table 1. Animals were kept for a 14-day observation period to evaluate a possible delayed toxicity and/or the reversibility of observed changes. Satellite groups were added to evaluate the immune response at Day 14 for both routes of administration (s.c. and i.m.) and at Days 1, 3 or 7 for the s.c. route of administration only.

TABLE 1
Group distribution of study

| | | | | | | | Dose- | Dose- |
|---|---|---|---|---|---|---|---|---|
| | | | | | Volume | | level | level |
| | | | | | of | | [μg of | [μg of |
| | Number of animals | | | Dosage form | injection | Route of | peptide/ | MPLA/ |
| Group | Total | Male | Female | administered | [mL] | administration | injection] | injection] |
| 1 | Principal = | 6 | 6 | PBS | 0.8 | s.c. | 0 | 0 |
| | 12 | | | | | | | |
| 2 | Principal = | 6 | 6 | ACI-24-250 | 0.2 | s.c. | 0 | 30 |
| | 12 | | | (empty) | | | | |
| 3 | Principal = | 6 | 6 | ACI-24-1000 | 0.8 | s.c. | 0 | 30 |
| | 12 | | | (empty) | | | | |
| | Satellite1 = | 5 | 5 | | | | | |
| | 10 | | | | | | | |
| 4 | Principal = | 7 | 6 | ACI-24-250 | 2 × 0.1 | i.m. | 0 | |
| | 11 | | | (empty) | | | | |
| 5 | Principal = | 6 | 6 | ACI-24-250 | 0.2 | s.c. | 65 | 30 |
| | 12 | | | | | | | |
| 6 | Principal = | 6 | 6 | ACI-24-1000 | 0.8 | s.c. | 260 | 30 |
| | 12 | | | | | | | |
| | Satellite1 = | 3 | 3 | | | | | |
| | 6 | | | | | | | |
| | Satellite2 = | 9 | 9 | | | | | |
| | 18 | | | | | | | |
| 7 | Principal = | 6 | 6 | ACI-24-1000-A | 0.8 | s.c. | 385 | 30 |
| | 12 | | | | | | | |
| 8 | Principal = | 6 | 6 | ACI-24-250 | 2 × 0.1 | i.m. | 2 × 32.5 | 30 |
| | 12 | | | | | | | |
| | Satellite1 = | 5 | 5 | | | | | |
| | 10 | | | | | | | |

Dose administered once at Day 0.
Blood sample for s.c. administration were collected on Days: 1, 3 or 7 and 14.
Blood sample for i.m. administration were collected on Day 14
ACI-24-250 and ACI-24-1000 corresponds to the targeted dose of the abeta1-15 antigen; 250 μg and 1000 μg respectively.

The animals were checked at least once daily for mortality and at least twice daily (three times on Day 1) for clinical signs. Skin reactions at injection site were recorded before injection, then 6, 24 and 48 hours, and then three and seven days after injection. The rectal temperatures were recorded before injection, then 6, 24 and 48 hours after injection and at the end of the observation period. Body weight and food consumption were recorded at least three times a week. Hematological and blood biochemical investigations were performed on, respectively, the three first principal animals and the three last principal animals, at the end of the observation period. Aβ1-42-specific IgG and IgM antibodies were determined by ELISA.

At the end of the observation period, all surviving animals were sacrificed and submitted to a full macroscopic post-mortem examination. The spleens of all satellite animals were sampled for separation of lymphocyte cells. Designated organs were weighed and selected tissue specimens were preserved for principal animals. Microscopic examination was conducted on subcutaneous injection sites of two satellite mice from Group 6 (total of nine male and nine female mice killed at 1, 3 and 7 days post-injection), stained with hematoxylin and eosin (HE) or with polyclonal rabbit anti-Aβ1-40 precursor protein termed thereafter Aβ.

Subsequent microscopic examination was performed on intramuscular injection sites (formalin-fixed muscle samples) of mice from Group 8 (6 males and 6 females), stained with hematoxylin-eosin.

### Results

The administration of ACI-24 once by s.c. (at the dose-levels of 65, 260 or 385 μg/injection) or i.m. route (at the dose-level of 65 μg/injection) to mice followed by an observation period of 14 days, was well tolerated. No deaths attributed to the treatment with vehicle or test item formulations were observed during the study period. No toxicologically relevant clinical signs and/or differences of rectal temperatures were attributed to the treatment with the test item.

No treatment-related skin reactions were noted.

The body weight and the food consumption were unaffected by the treatment with the test item. At laboratory investigations, no toxicologically relevant differences among hematological or biochemical parameters were observed in animals receiving the empty liposomes or the test item.

The microscopic examination of i.m. injection site showed that administration of ACI-24 (2×32.5 μg/injection) in the gastrocnemius muscle yielded in all treated mice minimal to slight non-adverse granulomatous inflammation after 2 weeks, characterized by mononuclear cell infiltrates associated with minimal fibrosis. These findings were considered to be non-adverse as the severity was of low magnitude.

### Conclusion

Under the experimental conditions of the study, the no observed adverse effect level (NOAEL) was established at 65 μg/injection by i.m. route and 385 μg/injection by s.c. route.

### 4.1.2 Evaluation of the Toxicity of ACI-24 Following Single-Dose Subcutaneous Administration in Monkeys

### Objective

The toxicity and local tolerance of a single subcutaneous injection of ACI-24 in cynomolgus monkeys was evaluated in this GLP study.

### Design

The study design is explained in Table 2.

TABLE 2
Group distribution of study

| | | | | | | Dose-level | Dose-level |
|---|---|---|---|---|---|---|---|
| | Number of | | | Volume of | | [μg of | [μg of |
| | animals | | Dosage form | injection | Route of | peptide/ | MPLA/ |
| Group | Male | Female | administered | [mL] | administration | injection] | injection] |
| 1 | 3 | 3 | PBS | 0.8 | s.c. | 0 | 0 |
| 2 | 3 | 3 | ACI-24-250 | 0.8 | s.c. | 0 | 128 |
| | | | (empty) | | | | |
| 3 | 3 | 3 | ACI-24-250 | 0.2 | s.c. | 96 | 9 |
| 4 | 3 | 3 | ACI-24-1000 | 0.8 | s.c. | 385 | 36 |

Dose administered once on Day 1.
Local tolerance evaluated after 6, 24, 48 hours and 7 days.
Rectal temperature recorded after 6, 24, 48 hours and 14 days.
ACI-24-250 and ACI-24-1000 corresponds to the targeted dose of the abeta1-15 antigen; 250 μg and 1000 μg respectively.

The dosage forms were administered once on Day 1. Clinical signs were evaluated, at least three times a day during the study and additionally approximately six hours after treatment on the day of treatment. The local tolerance at the injection site was evaluated on the day of treatment, before injection and 6, 24, and 48 hours and seven days after treatment. Rectal temperature was recorded on the day of treatment, before injection, 6, 24, and 48 hours after treatment and at the end of a 14-day observation period. The body weight of each animal was recorded at designated intervals and food consumption was estimated during the study. Electrocardiography examinations, blood pressure measurements and laboratory investigations (including hematology, blood biochemistry, urinalysis, blood lymphocyte subset analysis and seric immune response quantification) were performed during the pre-treatment period, after treatment and during the observation period. Ophthalmology examinations were performed during the pre-treatment period and once at the end of the 14-day observation period. On completion of the observation period, the animals were sacrificed for organ weight recording, macroscopic post-mortem examination and microscopic examination of selected tissues.

### Results

The administration of ACI-24 or empty liposomes once by s.c. injection to cynomolgus monkeys, was well tolerated. No unscheduled deaths occurred during the study. No systemic clinical signs were noted after treatment or during the observation period in any animal. There were no statistical differences in the body temperatures recorded between control and treated animals, at any time-point. The values recorded were within the range of normal values recorded in healthy animals of this strain and age. Body weight and food consumption were considered to be unaffected by the test item treatment.

Electrocardiography parameters, including PQ and QT intervals, QRS-complex duration and heart rate were unaffected by the test-item treatment. Systolic and diastolic blood pressure measurements were unaffected by the test item treatment at all time-points. No relevant ophthalmological findings were observed in any group during the pre-treatment period or at the end of the treatment period. Hematology parameters, including lymphocyte subset populations, blood biochemistry and urinalysis were not affected by the test item treatment at any time-points.

At necropsy, organ weights were not affected by the test item treatment and no systemic treatment-related macroscopic lesions were observed.

### Conclusion

The NOAEL following systemic single-dose administration of ACI-24 was considered to be 385 μg of peptide/injection under the experimental conditions of this study.

### 4.2 Repeated Dose Toxicity

4.2.1 Study to Assess the Potential Cross Reactivity of Cynomolgus Monkey Antibodies Against ACI-24 with a Selected Panel of Normal Human Tissues

### Objective

The objective of this GLP study was to assess the potential cross-reactivity of the serum antibodies from cynomolgus monkey treated with ACI-24 on histological cryostat sections of human tissues using immunohistochemical techniques.

### Design

The test material was a serum preparation from a cynomolgus monkey previously immunized with ACI-24 (Animal 6529, Day 31) injected at days 2 and 24 (bleeding at day 31, that was used for the immunostaining) with the vaccine ACI-24-250-another vaccine batch (Pal 1-15 antigen: 80 ug/dose target, MPLA: 30 ug/dose target). This serum contained anti-Amyloid (Aβ) IgG antibodies at an approximate concentration of 4 μg/mL. Serum from an empty liposome immunized monkey was used as negative control serum (Animal 6613, Day 49).

The test system used cryostat sections (5 μm thick) of human Alzheimer's brain tissue (Cortex) identified as being positive for the antibodies raised in Animal 6529, Day 31 (ACI-24 immunized monkey sera). Healthy human brain tissue (same region) was used as negative control. The system was validated by selecting tissue with a large number of small, distinct Amyloid plaques that were positive for Aβ screened with a mouse anti-Aβ antibody.

The detection method was validated by using serial dilutions of the test serum and negative control serum in order to determine the optimal dilution that yielded specific positive immunohistochemical staining with minimal non-specific background staining on human Alzheimer's and healthy brain tissues.

Cyrosections from a selected panel of human tissues (Table 3) were used for the assessment of potential tissue crossreactivity.

TABLE 3
Human tissue titration

| | | |
|---|---|---|
| Adrenal | Duodenum | Pituitary |
| Urinary Bladder | Ileum | Placenta |
| Blood Cells | Colon | Prostate |
| Bone Marrow | Heart | Skin |
| Breast | Kidney (glomerulus, | Spinal Cord |
| | tubule) | |
| Brain-Cerebellum | Liver | Spleen |
| Brain-Cortex | Lung | Striated Muscle |
| Endothelium | Lymph Node | Testis |
| Eye | Ovary | Thymus |
| Fallopian Tube | Pancreas | Thyroid |
| Oesophagus | Parathyroid | Tonsil |
| Gastric Antrum | Parotid | Ureter |
| Gastric body | Peripheral Nerve | Uterus (Cervix, |
| | | Endometrium) |

For each human tissue, three samples (from three donors) were used except lymph node and pituitary (2 samples) and parathyroid (1 sample).

### Results

Tissue viability was confirmed using anti-human antibodies against Vimentin, Von Willebrand Factor (Endothelial Marker), Cytokeratin and Transferrin Receptor (CD71).

In addition, a cryo-section from all tissues stained with Haematoxylin and Eosin indicated that there was no marked autolysis.

The titration results indicated that a 1:2000 dilution of serum 6529, Day 31 (ACI-24 immunized monkey sera) was optimal since there was specific staining seen in the Amyloid plaques and minimal nonspecific background staining of the surrounding tissue in human Alzheimer's brain tissue. No corresponding positive staining was seen in the human brain—Cortex negative control tissue. For the human tissue titration, the 1:2000 dilution and one lower (1:1000) and one higher (1:4000) dilution was used.

No specific positive staining was seen for serum 6529, Day 31 (ACI-24 immunized monkey sera) in any of the human tissues examined. Throughout the majority of tissues, this serum non-specifically stained smooth muscle cells (blood vessels, muscularis mucosae, and muscle layers), myoepithelial cells and other occasional stromal cells. Variable non-specific staining was seen in most of the tissues examined which was considered to be due to the use of the goat anti-monkey IgG antibody interacting with both the cynomolgus serum 6529, Day 31 (ACI-24 immunized monkey sera) and the negative control serum (Empty liposome immunized monkey sera). Although the intensity was higher with serum 6529, Day 31 (ACI-24 immunized monkey sera) than the negative control (Empty liposome immunized monkey sera), the location and distribution of the staining in serum 6613, Day 49 (Empty liposome immunized monkey sera) requires that it should be considered to be non-specific.

A minimal amount of non-specific staining was also seen in the buffer substitute negative control and is considered to be attributable to inadequate quenching of endogenous peroxidase in smooth muscle, connective tissue and macrophages. This minimal non-specific staining, considered to be endogenous peroxidase adds to that seen as a result of incubation with serum 6529, Day 31 (ACI-24 immunized monkey sera) and the negative control serum (Empty liposome immunized monkey sera).

### Conclusion

The results indicated that there was no specific positive staining attributable to the anti-ACI-24 antibodies in serum 6529, Day 31. It can therefore be concluded that cynomolgus monkey antibodies against ACI-24 do not cross react with human tissues.

### 4.2.2 Repeated-Dose Toxicity Following Subcutaneous Administration of ACI-24 in Cynomolgus Monkeys

### Objective

The objective of this study was to evaluate the potential toxicity of the test item, ACI-24, when administered to cynomolgus monkeys by the subcutaneous route every four weeks for a period of 21 weeks.

Upon completion of the treatment period, designated animals were held for a two week withdrawal period in order to evaluate the reversibility of any observed signs of toxicity.

Another objective of this study was to analyze the T-cell response induced by ACI-24 in the monkeys.

### Design

Two groups of three males and three females cynomolgus monkeys were treated once every four weeks, by the s.c. route, with the test item, ACI-24, at the dose levels of 28 μg (Group 3) or 78 μg (Group 4) of peptide/injection, with a total of six injections (21 weeks).

Five male and five female cynomolgus monkeys were treated at the dose-level of 311 μg (Group 5) of peptide/injection according to the same treatment design. Three males and three females (Group 2) were treated with ACI-24-empty and five males and five females (Group 1) were treated with PBS; both acting as control groups. Two animals/sex from Groups 1 and 5 were kept for a two-week recovery period.

TABLE 4
Group distribution of study

| | | | | | Dose-level | | Dose-level |
|---|---|---|---|---|---|---|---|
| | Number of | | | Volume of | [μg of | Peptide | [μg of |
| | animals | | Dosage form | injection/ | peptide/ | concentration | MPLA/ |
| Group | Male | Female | administered | animal | injection] | [μg/mL] | injection] |
| 1 | 5 | 5 | PBS | 0.8 mL | 0 | 0 | 0 |
| 2 | 3 | 3 | ACI-24- | 0.8 mL | 0 | 0 | 84 |
| | | | (empty) | | | | |
| 3 | 3 | 3 | ACI-24-30 | 0.2 mL | 28 | 142 | 9 |
| 4 | 3 | 3 | ACI-24-125 | 0.2 mL | 78 | 388 | 22 |
| 5 | 5 | 5 | ACI-24-500 | 0.8 mL | 311 | 388 | 88 |

Dose administered six times at following intervals: Week 1, 5, 9, 13, 17 and 21.
Blood sample for immunotoxicology withdrawn at following intervals: Week 15, 19 and 21.
Blood sample for immune response withdrawn every week (except week 1).
ACI-24-30, ACI-24-125 and ACI-24-500 corresponds to the targeted dose of the abeta1-15 antigen; namely 30 μg, 125 μg and 500 μg respectively

Blood samples for immunotoxicology were taken during the pre-treatment period, in Week 15, Week 19 and at the end of the treatment period. Blood samples for immune response analysis were taken weekly (except Week 1) from all the animals during the treatment period, and from the remaining animals of Groups 1 and 5 during the observation period. The animals were checked twice daily for mortality and clinical signs. Body weights were recorded twice during the pre-treatment period, on the first day of treatment and then once a week until the end of the study. Rectal temperature was taken before treatment (on the days of treatment) and 6, 24 and 48 hours after treatment. Additional measurements were taken at the end of the two-week observation period for the remaining animals in Groups 1 and 5. Rectal temperature was recorded on Day 15 for all animals. The food consumption was estimated daily throughout the study. Ophthalmological examinations were performed on all animals pretrial and on one occasion at the end of the treatment period. Electrocardiography examinations and blood pressure measurements were performed on all animals pretrial then at least two hours after the first dosing and on one occasion at the end of the treatment period.

Hematological investigations were performed on all animals pretrial then in Weeks 9, 15, 19, 21, 22 and at the end of the recovery period. Blood biochemistry analysis were performed on all animals pretrial then in Weeks 9 and 22 (end of treatment period) and at the end of the observation period. Urinalysis was performed pretrial and at the end of the treatment period. These examinations were also performed at the end of the observation period for the remaining of Group 1 and 5 animals.

Animals were submitted to a full macroscopic post-mortem examination. Designated organs were weighed and selected tissue specimens were preserved. A microscopic examination was performed on designated tissues from all animals sacrificed at the end of the treatment period.

To investigate the T-cell response, Peripheral Blood Mononuclear Cell (PBMCs) from monkeys treated with PBS, ACI-24-empty, ACI-24-30, ACI-24-125 or ACI-24-500 were pooled from Day 113 to Day 148 after the first immunization, corresponding to time points where antibody responses were observed. PBMCs were re-stimulated with Concanavalin A (positive control), Aβ1-42, Aβ1-15 or cell culture medium (negative control). The cells were pre-incubated with the stimulant for three hours and then transferred onto ELISPOT plates, where they were incubated for 48 h. The detection of IFN-γ, IL-4 and IL-5 producing cells was performed by an alkaline phosphatase-based detection system using an ELISPOT reader.

### Results

No unscheduled deaths or premature sacrifices occurred during the study. Thickening, edemas and nodules were observed with a dose-related severity at the injection sites and lasted for between 1-2 days and 1-2 weeks after administration of the dosage forms. Nodules were observed for one month in some animals, with no relationship to the dose-level administered. No local reactions were observed in control animals treated with PBS or animals treated with ACI-24-empty. Animals treated with active levels of test item showed slight to moderate local reactions at the injection sites.

The body weights and body weight gains were considered to be similar in control and treated animals during the treatment and observation periods. Food consumption was considered to be unaffected by the test item treatment. No ophthalmological alterations or electrocardiography findings were noted during the study in control or treated animals. Hematological and blood biochemistry parameters and urinalysis were considered to be unchanged at the different time-points evaluated.

The ACI-24 vaccine injected s.c. induced robust Aβ-specific IgG responses in five monkeys. The responding monkeys had been treated with ACI-24-30 (one monkey) ACI-24-125 (one monkey) or ACI-24-500 (three monkeys). Sustained anti-Aβ IgG titers were observed from Day 120 and onwards in three monkeys, suggesting that five immunizations were required to elicit an anti-Aβ IgG response in monkeys. Monkey treated with PBS or empty liposomes did not show any detectable anti-Aβ IgG antibodies as expected. Similar results were obtained when the Aβ-specific IgG response was measured in the plasma instead of the sera. ACI-24 induced anti-Aβ IgM titers in one of the monkeys receiving the highest dose (ACI-24-500). ACI-24 induced anti-MPLA IgG titers in two monkeys following ACI-24-30.

Complete reversibility was noted at the end of the observation period. At the injection sites, nodules and thickening of subcutaneous tissue correlated with s.c. granulomatous inflammation in all treated groups, including the vehicle control group (empty liposomes). Lesions in the vehicle control group were all of minimal severity. Minimal lesions in animals receiving active test item were similar in nature.

### Conclusion

Under the experimental conditions of the study, the NOAEL was established at 311 μg peptide/injection after six injections in cynomolgus monkeys, considering that the local reactions observed at the injection sites did not have an impact on the clinical status of the animals and were consistent with a normal granulomatous inflammatory reaction after s.c. injection of a foreign body.

This study also demonstrates that ACI-24 is capable of overcoming the immune tolerance to Aβ1-15 in monkeys.

The IL-4 results and the lack of correlation between IFN-γ secretion by PBMCs from monkeys immunized with ACI-24 and re-stimulation with Aβ1-15 together with the very low T-cell response indicate a preferential Th2 response for ACI-24 vaccine and thus a positive safety profile of ACI-24.

4.2.3 13-Week Toxicity Study by Subcutaneous Route in hAPP V717I Transgenic Mice

### Objective

The objective of this GLP compliant study was to evaluate the potential toxicity of ACI-24 in human Amyloid Precursor Protein over-expressing transgenic mice (hAPP V717I). The transgenic mouse model hAPP V71 was selected because it reflects the pathophysiology of patients with Aβ plaque deposits in the brain and is therefore, from a biological perspective, the most relevant model for the safety evaluation of ACI-24.

### Design

The hAPP V717I mice were immunized by subcutaneous administration of ACI-24 every two weeks for a total treatment period of 13 weeks. hAPP V717I mice were allocated to five different groups including three different doses of the peptide per injection (80, 160 and 400 μg; n=28) whereas PBS and empty liposomes (lacking the peptide antigen) served as negative controls (n=24). The study also examined the toxicity of MPLA integrated in liposomes in a dose of 100 μg MPLA per injection.

The study design is summarized in Table 5:

TABLE 5
Group distribution of study

| | Number | | Peptide | Volume of | μg Peptide | MPLA (ug) |
|---|---|---|---|---|---|---|
| | of | Test or | Concentration | Injection | per | per |
| Group | Animals | Control Items | [mg/mL] | [mL] | Injection | injection |
| 1 | 24 | PBS | 0 | 1.0 (2 × 0.5) | 0 | 0 |
| | females | | | | | |
| 2 | 24 | ACI-24-Empty | 0 | 1.0 (2 × 0.5) | 0 | 76 |
| | females | | | | | |
| 3 | 28 | ACI-24 | 0.4 | 0.2 | 80 | 15 |
| | females | | | | | |
| 4 | 28 | ACI-24 | 0.4 | 0.4 | 160 | 30 |
| | females | | | | | |
| 5 | 28 | ACI-24 | 0.4 | 1.0 (2 × 0.5) | 400 | 76 |
| | females | | | | | |

Dose administered seven times at following intervals: Day 1, Week 3, 5, 7, 9, 11 and 13.

### Results

ACI-24 immunization raised a dose-dependent humoral anti-Aβ immune response, characterized by mainly anti-Aβ IgGs and less anti-Aβ IgMs, but did not cause:
Treatment-related death
Enhanced incidence of mortality
Significant changes in clinical signs
Change in body weight or in relative or absolute organ weight
Dose-dependent changes in haematology and blood biochemistry. Some non-dose-dependent changes were considered of limited toxicological significance.

ACI-24 treatment led to minimal-to-moderate subcutis fibroplasia in the injections sites of all treated groups, with a minimal increase in incidence and severity in liposome treated groups (ACI-24 or empty liposomes), when compared to the PBS control group.

### T-Cell Response:

Splenocytes isolated from mice immunized with the high dose of ACI-24 (400 μg) and re-stimulated in vitro with Aβ1-15 peptide significantly increased the number of IL-4 secreting cells, suggesting that ACI-24 preferentially induces a Th2 response. No T-cell proliferation could be observed.

### Local Brain Inflammation:

Immunization with ACI-24 did not induce pro-inflammatory cytokine release (IFN-γ, TNF-α, IL-6) in brains of immunized mice but was associated with slightly decreasing levels of IFN-γ, TNF-α and IL-6.
Immunization with high doses of ACI-24 (400 μg) did not enhance the presence of T-cells (CD3, CD4 and CD8), macrophages (F4/80) nor B-cells (B220 or CD45R) in the brains of immunized mice as evaluated by immunohistochemistry.
Immunization with ACI-24 did not increase the incidence of micro-hemorrhages (Perl's hemosiderin) nor the severity of perivascular brown pigment-laden macrophages in the brain at any dose level, when compared to the PBS control group.
Immunization with ACI-24 did not change the density of vessels (collagen type-IV) nor enhance Thioflavin-S positive amyloid plaques in vessels, indicating that there is only a low risk of cerebral amyloid angiopathy (CAA).

### Conclusion

These data demonstrate that immunization with ACI-24 does not induce micro-haemorrhage, local brain inflammation, penetration of peripheral inflammatory cells (T-, B-cell or macrophages) nor perivascular accumulation of AP, indicating that ACI-24 as a vaccine does present a promising safety potential. As supported by the results of this study, the NOAEL (No Observed Adverse Effect Level) was set at 400 μg/injection of ACI-24 for systemic toxicity.

### 4.2.4 12-Week Subcutaneous Immunogenicity and Toxicity Study in the Cynomolgus Monkey

### Objective

The purpose of this GLP study was to assess the toxicity and immunogenicity of different batches of ACI-24 when administered once every two weeks for a total of five occasions, subcutaneously to cynomolgus monkeys.

### Design

The study design is explained in Table 6.

TABLE 6
Study design

| | Number of | | | Dosage | Peptide | MPLA |
|---|---|---|---|---|---|---|
| | animals | | Route of | volume | Dose- | dose |
| Group | Male | Female | administration | (mL/injection) | level (ug)* | (ug) |
| Group | 3 | 3 | s.c. | 3 | 0 (PBS) | 0 |
| 1 | | | | | | |
| Group | 3 | 3 | | 3 | 1323 μg/inject. | 204 |
| 2 | | | | | (Old, | |
| | | | | | comparator)* | |
| Group | 3 | 3 | | 2 | 880 μg/inject. | 214 |
| 3 | | | | | (New, 6.9 | |
| | | | | | batch)* | |
| Group | 3 | 3 | | | 1320 μg/inject. | 321 |
| 4 | | | | | (New, 6.9 | |
| | | | | | batch)* | |
| Group | 3 | 3 | | 3 | 1278 μg/inject. | 207 |
| 5 | | | | | (New, 7.4 | |
| | | | | | batch)* | |

*The details given refer to changes in the manufacturing techniques utilized to produce the various batches. Group 2 were administered with the batch previously assessed in toxicological studies and was therefore used as a comparator group. Groups 3, 4 and 5 were administered the additional batches produced under revised manufacturing conditions which lead to a limited the hydrolysis of the MPLA during the first steps of the manufacturing process (as described in WO2012/055933, incorporated herein by reference). In addition the pH of the final solution was decreased from 7.4 to 6.5 to improve the stability of MPLA during storage.

Dose administered seven times at following intervals: Day 1, 15, 29, 43 and 57.
Blood sample withdrawn at following intervals: Pre-dose, Day 15, 29, 43, 57 and 71.

Throughout the study, all animals were observed at least twice daily for viability/mortality and clinical signs. Injection areas were observed daily during treatment and recovery periods.

Food consumption was estimated for each cage (qualitatively) twice daily during the study.

All animals were weighed twice a week during pretest and then weekly during treatment and recovery periods.

Blood samples were collected for clinical laboratory investigations during pretest and at the end of the treatment period (Week 12).

Blood samples were taken for IgG anti-Abeta determination once during pretest and 14 days after each administration.

At termination blood samples were collected to obtain serum, plasma and PBMCs which were either stored or analysed as part of a separate study.

Following completion of the scheduled treatment period, animals from Groups 1, 3 and 4 were necropsied and various organs were weighed. Macroscopic alterations were recorded. A full set of tissues and organs were collected, processed and examined histologically. Animals from Groups 2 and 5 were retained for future investigation work and therefore subsequently removed from the study.

### Results

No death occurred during the study protocol. There were no relevant clinical signs or local effects at the injection sites. Neither effects on food consumption nor on body weight occurred throughout the treatment period.

Subcutaneous administration of three formulations of ACI-24 induced a comparable profile in anti-Aβ IgG antibody across all groups, therefore indicating suitable correlation between the batches. One animal vaccinated with ACI-24 “New 6.9 batch” (Group 3 Female 24), showed a sustained anti-Aβ IgG titers from Day 43 onwards that were three-fold higher than those typically seen. Monkeys dosed with PBS did not show any detectable anti-Aβ IgG antibodies, as expected.

There were no relevant changes in the hematology or blood chemistry parameters.

No relevant macroscopic findings or noteworthy changes in the organ weights were recorded at necropsy.

Histological findings at the injection sites consisted of mononuclear cell focus/foci in subcutaneous tissue, with an increased incidence in Group 3 and increased incidence and severity in Group 4. These findings were present in monkeys of all groups examined (1, 3 and 4), including one control male. These changes were of minimal-slight intensity and their distribution was strictly local.

### Conclusion

Five subcutaneous administration, once every two weeks, using different batches of ACI-24, to cynomolgus monkeys (up to approximately 1320 μg/injection), was well tolerated with no effects on body weight, food consumption or clinical pathology parameters.

Based on the results obtained and under these study conditions, all batches of ACI-24 assessed were considered comparable in terms of toxicity and immunogenicity with an approximate dose of 1320 μg/injection currently considered as a NOAEL (No Observed Adverse Effects Level).

### Example 5: A Phase 2 Double-Blind, Randomized, Placebo-Controlled Study to Assess the Safety, Tolerability and Target Engagement of ACI-24 in Adults with Down Syndrome

### Primary Outcome Measures:

Number of participants with Adverse Events (AEs) assessed by intensity (mild, moderate or severe) and causal relationship (unrelated, unlikely, possibly or probably related)
[Time Frame: from screening up to week 100]
Mean change from baseline in vital signs
systolic and diastolic blood pressure (mmHg), hear rate (bpm), body temperature (degree Celsius)
[Time Frame: from baseline up to week 100]
Mean change from baseline in suicidal ideation/behavior using Columbia-Suicide Severity Rating Scale (C-SSRS)
[Time Frame: from baseline up to week 100]
Number of participants reporting suicidal ideation or behavior using Columbia-Suicide Severity Rating Scale (C-SSRS) [Time Frame: from baseline up to week 100]
Number of participants with abnormal MRI results
Occurrence of Amyloid-related imaging abnormalities (ARIA)
[Time Frame: from baseline up to week 100]

### Secondary Outcome Measures:

Change from baseline of composite standardized uptake value ratio (SUVR) assessed by amyloid PET imaging using florbetaben
[Time Frame: from baseline up to week 76]
Change from baseline in anti-Aβ antibody titers in blood
[Time Frame: from baseline up to week 100]
Change from baseline of amyloid-related biomarkers (Aβ1-40, Aβ1-42), total tau, phosphorylated tau and NfL in blood/CSF (pg/mL) (CSF is optional).
[Time Frame: from baseline up to week 100]
Change from baseline of brain tau load assessed by tau PET imaging
[Time Frame: from screening up to week 74]
Change from baseline of cognitive performance using Cambridge Neuropsychological Test Automated Battery—Paired Associates Learning [CANTAB-PAL]
Score is a z-score ranging from −7.5 to 0. A higher score (eg., 0) indicates a better outcome.
[Time Frame: from baseline up to week 100]
Change from baseline of cognitive performance using Cambridge Cognitive Examination—Down Syndrome [CAMCOG-DS]
[Time Frame: from baseline up to week 100]
The total score ranges from 0 to 107. A higher score indicates a better outcome.
Change from baseline in adaptive behavior (Vineland Adaptive Behavior Scale)
[Time Frame: from baseline up to week 100]
The composite score ranges from 20 to 140. A higher score indicates a better outcome.
Change from baseline in Clinical Global Impression of Change (CGIC)
[Time Frame: from baseline up to week 100]
The score ranges from 1 to 7. A higher score indicates a worse outcome.

### Method:

This study is a prospective multicenter, placebo-controlled, double-blind, randomized study to assess the effect of one dose of the ACI-24 vaccine, versus placebo over a 74-week treatment period and 26-week safety follow-up period.

After the screening period, eligible subjects are randomized in a 1:1 ratio to receive either ACI-24 or corresponding placebo, both given by the intramuscular route. Approximately 72 subjects (36 subjects receiving ACI-24 1000 μg and 36 subjects receiving placebo) are randomized in the study.

Subjects are treated with repeated administrations of ACI-24 (1000 μg dose) or corresponding placebo using the intramuscular route. ACI-24 (1000 μg dose) or placebo is administered 8 times (each time, the dose of study treatment is administered in 2 separate concomitant intramuscular injections): the first 4 administrations are at 4-week intervals (W0, W4, W8, and W12); the next 3 administrations are at 12-week intervals (W24, W36, and W48); and the last administration is at W74 (26-week interval from previous administration). The 74-week treatment period is followed by a 26-week safety follow-up period.

### Inclusion Criteria:

Male or female subjects with DS with a cytogenetic diagnosis being either trisomy 21 or complete unbalanced translocation of the chromosome 21.
Age ≥40 and ≤50 years at screening.
Elevated brain Aβ as evidenced by composite SUVR ≥1.25 on florbetaben PET scan assessed by central reading.
Subjects, their legal representatives (if applicable) and/or their study partners in the opinion of the investigator, are able to understand and to provide written informed consent before starting any study-related activities.
In the opinion of the investigator, subjects, their legal representatives (if applicable) and/or their study partners are able to fully participate in the study, be sufficiently proficient in the official languages(s) of the country they are living in, and be capable of reliably completing study assessments.
Mild to moderate intellectual disability as per Diagnostic and Statistical Manual of Mental Disorders (DSM-5) classification.
Subjects must have a study partner who has direct and regular contact with the subject and who is able to provide reliable answers to questions related to the subject, according to the study investigator.
Subjects in preclinical stage of AD or with mild cognitive impairment due to AD.

### REFERENCE UST

Belichenko P V, Madani R, Rey-Bellet L, et al. An Anti-β-Amyloid Vaccine for Treating Cognitive Deficits in a Mouse Model of Down Syndrome. PLOS ONE. 2016; 11(3):e0152471.
Folstein M F, Folstein S E, McHugh P R (1975) “Mini-Mental State”: a practical method for grading the cognitive state of patients for the clinician J Psychiatr Res 12: 189-198
Gilman S., Koller M., Black R. S., Jenkins L., Griffith S. G., Fox N. C., Eisner L., Kirby L., Boada Rovira M., Forette F., Orgogozo J. M., Clinical effect of Aβ immunization (AN1792) in patients with AD in an interrupted trial. Neurology 64, 1553-1562 (2005).
Hartley S L, Handen B L, Devenny D, et al. Cognitive decline and brain amyloid-β accumulation across 3 years in adults with Down syndrome. Neurobiology of aging. 2017; 58:68-76.
Head E, Powell D, Gold B T, Schmitt F A. Alzheimer's Disease in Down Syndrome. European journal of neurodegenerative disease. 2012; 1(3):353-364.
Hughes C P, Berg L, Danzinger W L et al (1982) A new clinical scale for the staging of dementia. Am J Psychiatry; 140: 566-572
Monsonego A., Weiner H. L., Immunotherapeutic approaches to Alzheimer's disease. Science. 31; 302(5646):834-8 (2003).
Muhs A., Hickman D. T., Pihlgren M., Chuard N., Giriens V., Meerschman C., van der Auwera I., van Leuven F., Sugawara M., Weingertner M.-C., Bechinger B., Greferath R., Kolonko N., Nagel-Steger L., Riesner D., Brady R. O., Pfeifer A., Nicolau C., Liposomal vaccines with conformation-specific amyloid peptide antigens define immune response and efficacy in APP transgenic mice. PNAS, 104 23:9810-9815 (2007).
Nasreddine Z S, Phillips N A, et al. The Montreal Cognitive Assessment, MoCA: A brief screening tool for mild cognitive impairment. J Am Geriatr Soc. 2005; 53:695-699.
Orgogozo J. M., Gilman S., Dartigues J. F., Laurent B., Puel M., Kirby L. C., Jouanny P., Dubois B., Eisner L., Flitman S., Michel B. F., Boada M., Frank A., Hock C., Subacute meningoencephalitis in a subset of patients with AD after Abet42 immunization. Neurology 61: 46-54 (2003).
Prasher V P, Huxley A, Haque M S (2002) A 24-week, doubleblind, placebo-controlled trial of donepezil in patients with Down syndrome and Alzheimer's disease—pilot study. Int J Geriatr Psychiatry 17(3):270-278 (PMID: 11921156)
Pihlgren M., Silva A. B., Madani R., Giriens V., Waeckerle-Men Y., Fettelschoss A., Hickman D. T., López-Deber M. P., Ndao D. M., Vukicevic M., Buccarello A. L., Gafner V., Chuard N., Reis P., Piorkowska K., Pfeifer A., Kundig T. M., Muhs A., Johansen P., TLR4- and TRIF-dependent stimulation of B lymphocytes by peptide liposomes enables T cell-independent isotype switch in mice. Blood. January 3; 121(1):85-94 (2013).
Soto C., Plaque busters: strategies to inhibit amyloid formation in Alzheimer's disease. Molecular Medicine Today (vol 5), August 1999.
Winblad B., Graf A., Riviere M. E., Andreasen N., Ryan J. M., Active immunotherapy options for Alzheimer's disease. Alzheimers Res Ther. 2014 Jan. 30; 6(1):7.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

1. A liposomal vaccine composition comprising:
a. A β-amyloid (Aβ)-derived peptide antigen displayed on the surface of the liposome that comprises, consists essentially of or consists of amino acids 1-15 of Aβ; and
b. An adjuvant comprising monophosphoryl lipid A (MPLA)
for use in inducing an anti-Aβ immune response in a human subject without inducing a serious adverse event, wherein the β-amyloid (Aβ)-derived peptide antigen is administered in an amount of 300-2000 μg.

2. The liposomal vaccine composition for use of claim 1 wherein the β-amyloid (Aβ)-derived peptide antigen is administered in an amount of 500-2000 μg, preferably 1000-1500 μg, more preferably in an amount of 1000 μg.

3. The liposomal vaccine composition for use of claim 1 or 2 wherein the MPLA is administered in an amount of 15-600 μg, such as 50-600 μg, preferably 150-450 μg and more preferably 175 or 225 μg.

4. The liposomal vaccine composition for use of any one of claims 1 to 3 wherein the β-amyloid (Aβ)-derived peptide antigen is administered in an amount between 850 and 1150 μg, preferably 1000 μg and the MPLA adjuvant is administered in an amount of between 50 and 300 μg, preferably 175 or 225 μg.

5. The liposomal vaccine composition for use of any one of claims 1 to 3 wherein the β-amyloid (Aβ)-derived peptide antigen is administered in an amount between 255 and 345 μg, preferably 300 μg and the MPLA adjuvant is administered in an amount of between 15 and 90 μg, preferably 52.5 or 67.5 μg.

6. The liposomal vaccine composition for use of any one of claims 1 to 5 wherein the β-amyloid (Aβ)-derived peptide antigen is lipidated.

7. The liposomal vaccine composition for use of any one of claims 1 to 6 wherein the β-amyloid (Aβ)-derived peptide antigen is tetrapalmitoylated.

8. The liposomal vaccine composition for use of any one of claims 1 to 7 wherein the adjuvant forms part of the outer layer of the liposome, optionally wherein the adjuvant is, at least in part, displayed on the surface of the liposome.

9. The liposomal vaccine composition for use of any one of claims 1 to 8 wherein the monophosphoryl lipid A (MPLA) comprises synthetic monophosphoryl lipid A (MPLA).

10. The liposomal vaccine composition for use of claim 9 wherein the monophosphoryl lipid A (MPLA) comprises monophosphoryl Hexa-acyl Lipid A, 3-Deacyl (Synthetic) (3D-(6-acyl) PHAD®) and/or Phosphorylated HexaAcyl Disaccharide (PHAD®).

11. The liposomal vaccine composition for use of any one of claims 1 to 10 wherein the liposome comprises phospholipids.

12. The liposomal vaccine composition for use of any one of claims 1 to 11 wherein the phospholipids comprise dimyrsitoylphosphatidyl-choline (DMPC) and dimyrsitoylphosphatidyl-glycerol (DMPG).

13. The liposomal vaccine composition for use of any one of claims 1 to 12 wherein the liposome comprises cholesterol.

14. The liposomal vaccine composition for use of claim 13 wherein the molar ratio of dimyrsitoylphosphatidyl-choline (DMPC):dimyrsitoylphosphatidyl-glycerol (DMPG):cholesterol is 9:1:7.

15. The liposomal vaccine composition for use of claim 14 wherein the molar ratio of dimyrsitoylphosphatidyl-choline (DMPC):dimyrsitoylphosphatidyl-glycerol (DMPG):cholesterol:MPLA is 9:1:7:0.05.

16. The liposomal vaccine composition for use of any one of claims 1 to 15 wherein the liposomal vaccine composition is administered by injection.

17. The liposomal vaccine composition for use of any one of claims 1 to 16 wherein the liposomal vaccine composition is administered intramuscularly or subcutaneously.

18. The liposomal vaccine composition for use of claim 17 wherein the liposomal vaccine composition is administered intramuscularly.

19. The liposomal vaccine composition for use of claim 17 wherein the liposomal vaccine composition is administered subcutaneously.

20. The liposomal vaccine composition for use of any one of claims 1 to 19 wherein the liposomal vaccine composition is administered at a first time and is administered at a second time 1 to 4 weeks later.

21. The liposomal vaccine composition for use of any one of claims 1 to 20 wherein the liposomal vaccine composition is administered every 4-12 weeks for a period of at least 48 weeks, preferably wherein the liposomal vaccine composition is administered every 4 weeks for a period of 12 weeks and every 12 weeks for a further period of at least 36 weeks.

22. The liposomal vaccine composition for use of any one of claims 1 to 21 further comprising a booster administration at a subsequent time point.

23. The liposomal vaccine composition for use of any one of claims 1 to 22 wherein the induced anti-Aβ immune response is for treatment, prevention, induction of a protective immune response against or alleviating the symptoms associated with an amyloid-beta associated disease or condition in the human subject.

24. The liposomal vaccine composition for use of claim 23 wherein the amyloid-beta associated disease or condition is selected from Alzheimer's Disease, mild cognitive impairment (MCI), Down syndrome (DS), including Down syndrome-related Alzheimer's disease, cardiac amyloidosis, cerebral amyloid angiopathy (CAA), multiple sclerosis, Parkinson's disease, Lewy body dementia, ALS (amyotrophic lateral sclerosis), Adult Onset Diabetes, inclusion body myositis (IBM), ocular amyloidosis, glaucoma, macular degeneration, lattice dystrophy and optic neuritis.

25. The liposomal vaccine composition for use of claim 24 wherein the amyloid-beta associated disease or condition is Alzheimer's Disease.

26. The liposomal vaccine composition for use of claim 25 wherein the Alzheimer's Disease is early Alzheimer's Disease.

27. The liposomal vaccine composition for use of claim 26 wherein the early Alzheimer's Disease includes mild cognitive impairment due to Alzheimer's Disease and mild Alzheimer's Disease.

28. The liposomal vaccine composition for use of claim 25 wherein the Alzheimer's Disease is mild Alzheimer's Disease.

29. The liposomal vaccine composition for use of claim 25 wherein the Alzheimer's Disease is mild-to-moderate Alzheimer's Disease.

30. The liposomal vaccine composition for use of claim 25 wherein the Alzheimer's Disease is moderate Alzheimer's Disease.

31. The liposomal vaccine composition for use of claim 25 wherein the Alzheimer's Disease is not severe Alzheimer's Disease.

32. The liposomal vaccine composition for use of claim 24 wherein the amyloid-beta associated disease or condition is Down Syndrome.

33. The liposomal vaccine composition for use of claim 24 or 32 wherein the amyloid-beta associated disease or condition is Down syndrome-related Alzheimer's disease.

34. The liposomal vaccine composition for use of any one of claims 1 to 33 wherein the human subject, prior to treatment, displays cognitive function consistent with a Mini Mental State Examination (MMSE) score of at least 18, such as 18-28, or at least 20, such as 20-28.

35. The liposomal vaccine composition for use of any one of claims 1 to 34 wherein the β-amyloid (Aβ)-derived peptide antigen is tetrapalmitoylated Abeta 1-15 as set forth in SEQ ID NO: 1.

36. The liposomal vaccine composition for use of any one of claims 1 to 35 wherein the administered amount of Abeta 1-15 as set forth in SEQ ID NO: 2 is 152-1016 μg.


SEQUENCE LISTING (2 sequences)

SEQ ID NO: 1 - protein, 19 aa, Artificial sequence
  Tetrapalmitoylated Abeta 1-15
  Lys (1)..(2): Each Lys is palmitoylated
  Lys (18)..(19): Each Lys is palmitoylated
KKDAEFRHDSGYEVHHQKK

SEQ ID NO: 2 - protein, 15 aa, Artificial sequence
  Synthesised Abeta 1-15
DAEFRHDSGYEVHHQ